SEQUENCE LISTING (14 sequences)

SEQ ID NO: 1 - DNA, 726 bp, Homo sapiens
  CDS (1)..(726): human pre-pro-NGF
atgtccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 2 - protein, 241 aa, Homo sapiens
MSMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 3 - protein, 18 aa, Homo sapiens
MSMLFYTLITAFLIGIQA

SEQ ID NO: 4 - protein, 103 aa, Homo sapiens
EPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKR

SEQ ID NO: 5 - DNA, 2095 bp, Artificial Sequence
  synthetic expression cassette for the expression in plant of rh pre-pro-NGF
aagcttgcatgcctgcaggtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgataacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattcacaacacaaatcagatttatagagagatttataaaaaaaaaaaaacatatggagtggagctggatctttctctttctcctctcaggaactgcaggtgttcactccatgaccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccgtcgacgaaaatctttattttcaaggagtcgacaaggatgaacttcatcaccatcatcaccattaactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtaccggcgcgcctctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagctcctagctagcatcgac

SEQ ID NO: 6 - DNA, 1920 bp, Artificial Sequence
  synthetic expression cassette for the expression in plant of hr pre-pro-NGF
aagcttgcatgcctgcaggtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgataacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattcacaacacaaatcagatttatagagagatttataaaaaaaaaaaaacatatggagtggagctggatctttctctttctcctctcaggaactgcaggtgttcactccatgaccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccgtcgacggaggtggaggttctgcggccgctcgtggatctgagaaagatgagctctaaactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtaccggcgcgcctctagagtccgcaaaaatcaccagtctctctctacaaatctatctctctctatttttctccagaataatgtgtgagtagttcc

SEQ ID NO: 7 - DNA, 2068 bp, Artificial Sequence
  synthetic expression cassette for the expression in plant of hr pre-pro-NGF
aagcttgcatgcctgcaggtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgataacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaattcacaacacaaatcagatttatagagagatttataaaaaaaaaaaaacatatggagtggagctggatctttctctttctcctctcaggaactgcaggtgttcactccatgaccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccgtcgacaaggatgaacttcatcaccatcatcaccattaactcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccaggtaccggcgcgcctctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcaggcatgcaagctcctagctagcatcgac

SEQ ID NO: 8 - DNA, 76 bp, Artificial Sequence
  synthetic oligo for the insertion of TEV-KDEL-6XHis-Tag
tcgacgaaaatctttattttcaaggagtcgacaaggatgaacttcatcaccatcatcaccattaactcgaggagct

SEQ ID NO: 9 - DNA, 23 bp, Artificial Sequence
  synthetic pNGF sense primer
cccaataacagttttaccaaggg

SEQ ID NO: 10 - DNA, 21 bp, Artificial Sequence
  synthetic pNGF antisense primer
caggtcaggctcttctcacag

SEQ ID NO: 11 - DNA, 28 bp, Artificial Sequence
  synthetic pNGF sense Bsph primer
cctcatgaccatgttgttctacactctg

SEQ ID NO: 12 - DNA, 29 bp, Artificial Sequence
  synthetic pNGF antisense Sal primer
atagtcgacggctcttctcacagccttcc

SEQ ID NO: 13 - DNA, 21 bp, Artificial Sequence
  synthetic p35S sense primer
cactgacgtaagggatgacgc

SEQ ID NO: 14 - protein, 7 aa, Artificial Sequence
  KDEL recognition site
ENLYFQG

(19) United States
(12) Patent Application Publication
(10) Pub. No.: US 2011/0213132 A1
(43) Pub. Date: Sep. 1, 2011

(54) PRODUCTION OF NGF IN PLANT

(76) Inventors: Piera Galba, Milano MI (IT); Carlo Massimo Pozzi, Milano (IT); Maria Rosaria Stile, Lodi LO (IT); Enrico Puja, Lodi LO (IT); Elena Audia, Lodi Lo (IT)

(21) Appl. No.: 13/121,556

(22) PCT Filed: Jul. 2, 2009
(86) PCT No.: PCT/IB09/52877

§ 371 Date: Mar. 29, 2011

(30) Foreign Application Priority Data
Sep. 30, 2008 (IT) RM2008A000517

(51) Int. Cl. C07K 1/36; A01H 5/00; C12N 5/10; A01H 5/12; A01H 5/04; A01H 5/02; A01H 5/10; A01H 5/06; A01H 1/06; C12P 21/06; C07K 14/48

(52) U.S. Cl. 530/399; 800/298; 435/419; 800/288; 435/68.1

(57) ABSTRACT

The invention relates to genetically modified plants capable of producing a human recombinant protein Nerve Growth Factor, either in the form of pre-pro-protein or in the mature form and parts and differentiated and undifferentiated tissues thereof. The invention relates, also, to methods for the transformation of said plants in a transient way and methods for the transformation of said plants and tissues in a stable or transient way, methods for the recombinant protein purification from crude extract of proteins derived from plant tissue of said plants.

The invention relates to genetically modified plants capable of producing a human recombinant protein Nerve Growth Factor, either in the form of pre-pro-protein or in the mature form and parts and differentiated and undifferentiated tissues thereof.

The invention relates, also, to methods for the transformation of said plants in a transient way and methods for the transformation of said plants and tissues in a stable or transient way, methods for the recombinant protein purification from crude extract of proteins derived from plant tissue of said plants.

### STATE OF THE ART

The Nerve Growth factor (NGF), factor for the growth of nerves, is a signal protein involved in the development of the nervous system in vertebrates. It directs and regulates the growth of axons through mechanisms of cells signalling.

NGF was discovered in the 50 by Rita Levi Montalcini, who carried out researches on this protein molecule and on its mechanism of action, for which, in 1986, she won the Nobel Prize for medicine together with the US citizen Stanley Cohen.

The human NGF, obviously, cannot be produced by extraction from human tissues, consequently, many different techniques for rh-NGF (i.e. Recombinant human NGF) production have been developed, and that often did not bring to an effective production of the molecule.

Recombinant DNA technologies allow the production a wide range of human recombinant proteins for therapeutic applications; however the various attempts for the production of recombinant NGF using different organisms have not yet given the expected results.

Herein are reported the systems actually available to produce recombinant human NGF.

Production of rhNGF in Eisenia Foetida (WO/2006/082609 human NGF-like protein isolated from Eisenia foetida and uses thereof). This method is very toilsome and not easy to industry scale-up because it requires the growing of several copies of Eisenia foetida, which requires the use of expensive system of growth and conservation of the bioreactor (insect cell, mammalian cell, or micro-organisms) and long steps for the preparation of the annelid before proceeding with the extraction of the recombinant protein. The use of animals to produce the protein requires also the use of sterility condition maintenance systems and does not allow the continuous production of molecule of interest.

Production of rhNGF in micro-organisms (U.S. No. 594126 1990-10-09 Production of biologically active, recombinant members of the NGF/BDGF family of neurotrophic proteins).This method has the disadvantage of using a prokaryotic system which is very different from the eukaryotic system in which the NGF production occurs physiologically, this difference has as a consequence that, for the production of Eukaryotic proteins with a correct folding, several toilsome refolding steps in vitro are required after the purification step. In addition the bacterial system requires the presence of bio-fermenters that hardly work in continuous and that require high and expensive sterility conditions.

By way of example, although the large number of models for heterologous proteins production in prokaryotic systems, the failure in the rhNGF production in E. coli was found to depend on the inability of the bacteria to process correctly the NGF precursor making the correct proteolytic cuts Moreover, also the expression of the DNA region coding for the mature protein leads to the synthesis of a biologically inactive protein that forms inclusion bodies. The absence of biological activity is due to the absence of the correct disulfide bonds formation which is not favoured in the reducing prokaryotic cytosol. Furthermore, the solubilisation of the inclusion bodies and the refolding of protein in its tertiary biologically active structure brings, anyway, a low yield of production and low biologically activity in comparison with high production and purification cost.

The rhNGF production was obtained also in insect cells (Barnett et al. J. Neurochem. 57, 1052-1061, 1991, U.S. Pat. No. 5,272,063) and in mammalian cells (Iwane et al. Biochem. Biophys. Res Commun. 171,116-122, 1990; U.S. Pat. No. 5,639,664; Grey, Genetech U.S. Pat. No. 5,288,622) with better results in the yield of in vitro biologically active recombinant protein production.

The rhNGF was also produced, in an effective way and in an active form, also in vivo in mammalian cells as described in patent application WO2007034266. Nevertheless, all the procedures since now described are, either not effective (cfr. prokaryotic systems) or, however, expensive as they need a molecule production in bio-reactors and require expensive systems for sterility maintenance.

Some production system of growth factor in plant have been suggested (described as possible systems but without any proof or evidence of production) which foresee the use of engineered monocotyledon seeds such as rice, barley, triticum and sorghum, engineered (US2004/0078851 Al production of human Growth Factors in monocot seeds).

This system foresees the use of expression vectors in plant having a seed specific promoter, which implies that the recombinant protein is only expressed in monocot seeds. The problem in this case derives from the localisation of the protein in the seed that, although being an effective storage system, presents some problems indicated below in the extraction and purification of the recombinant protein phase and in general of all the endogenous proteins.

In the patent application indicated above dicotyledonous seeds such as tobacco etc. are used. It is well known that the seeds of those varieties have dimension such as a pinhead, hence, to reach an amount of seeds needed for a large scale production it would be necessary to farm in retaining conditions numerous hectares of soil.

The hypothesis that up to date it would be possible to farm a huge number of hectares of soil with OGM is unfeasible.

Moreover, in order to extract from seed proteins is necessary to finely grind the starting materials. Therefore is necessary to use a very big mill that must be in right sterility conditions. To date such machines are not known in the art, i.e. mills normally used for flours do not respond sufficiently to the needs of working under sterility conditions.

Another practical problem is that the seed is rich in wood components that require a filtration step, for the purification of hexogen protein expressed in seeds, with sieves suitable for retain such materials.

The seed is also rich in fat and oily substances, which make impossible any step in chromatography because every matrix used in chromatography are saturated by these substances, so a further step (chemical) is necessary for the removal of fat substances.

Only after making these steps for the preparation of the total protein extract is possible to envisage carrying out the real purification steps.

In said patent application discussed hence, plants are indicated and used, for which, to date, a hypothetic producer of the protein of interest, would find many problems in various countries due to actual government positions and to the public opinion about safety of GMO plants normally used for alimentary use are suggested and the farming of those plants in field would bring to pollen cross-contamination of cultivars of the same species used for food and not for pharmaceutical production. Moreover, in the above patent application, even if NGF expression is claimed, there is no concrete example that teaches how to carry out the production of recombinant human NGF (rhNGF). The only proteins expressed in the description are EGF, IGF, and ITF, nothing is exemplified for NGF.

It is clear that the systems proposed and suggested are not really suitable for rhNGF production. Moreover, a fact not negligible at all, is that at the present state of the art, the systems proposed up to date for the purification of nervous grown factors (WO patent 98/21234-purification of neurotrophins) are not suitable for the correct purification of NGF derived from vegetal tissues due to the presence, inside the plant tissues, of substances like pigments, waxes, wooden material. The purification systems proposed in said patent application WO 98/21234(purification of neurotrophins) are not applicable for purification from complex matrixes such as plant matrix. The method suggested in said patent application would not lead to positive results or would be limited to extremely low yields (less than 50%) if applied to plant systems. The system proposed in the patent application above, in fact, not foresee the use of methods, that are necessary in plants, for the removal from the crude extract of molecules or of aspecific substances that due to weak interactions (ionic bond, Van der Walls bond) or due to their relative abundance, would saturate the sites of the stationary stage of the chromatographic column.

In plant, among the substances mainly interfering with a good chromatography result are:

the pigments, that are coloured macro-molecules (e.g. chlorophyll) that may be more or less polar, which can often cause non-specific bonds thereof to chromatographic matrix that saturating them. This implies an inaccessibility of the recombinant proteins, objects of the purification, to the matrix with the consequence that the same are eliminated by filtering. In addition, it has to be noted that, pigments, by their own nature, interfere also with the principal detection systems used for the following of the purification steps. These systems, in fact, are based on the recombinant protein absorbance at a specific wavelength, the presence of coloured substances such as pigments renders the data unreliable.

Wax and fat substances; for their own nature said substances transform the crude extract in a less fluid extract, this effect renders the extract less capable of flowing through the chromatographic column with a subsequent saturation of the matrix active sites.

The facts reported in literature highlight that to date systems that are at the same time effective, low cost and/or of easy industrial scaling up for the production of human pre-proNGF and/or recombinant human NGF do not exist.

In fact, all the systems proposed for the protein production are affected by side effects rendering an industrial production difficult or expensive, or rendering impossible to obtain the authorization to the commercialisation of that product, or that may cause the production of a protein with an incorrect folding and hence incapable of performing the function of active principle in pharmaceutical applications.

It would hence be desirable to be able to standardize an rhNGF production in a way at the same time effective, bringing to the production of a product with the right molecular folding, repeatable, that would allow a low cost in continuous production.

### SUMMARY OF THE INVENTION

In the present description a new method for the production of rhNGF, having the advantages of being in an eukaryotic system and hence to produce rhNGF that does not require several steps of in vitro refolding of the purified protein, that does not require the use of bio-fermenters, that does not require the use of expensive systems of growth and maintenance of the bioreactor (as it happens for the cultured cells), that does not require the use of systems for the maintenance of the sterility conditions, that allows to work in continuous and being quite economical is described.

The present description teaches the realisation of an effective system, cost-effective and of immediate industrial scaling-up for the production of the human recombinant protein NGF in the form of pre-pro-protein and in the form of mature protein. For the carrying out of the invention a technology of plant molecular farming, which involves the use of plants as bioreactors for the production of recombinant proteins has been used.

The present invention relates to the engineering of dicotyledon plants in a stable way and in a transient way, through the use of expression vectors bringing the gene coding for the human protein pre-pro-NGF, for obtaining the production of pre-pro-NGF and of NGF in plants, said engineered plants and parts of said plants. In order to avoid, as previously said, the possibility of “contaminating” edible plants grown with feeding intent with the genetically modified plant herein described, it a dicotyledon plant not grown for feeding has been used.

The description also teaches new systems for the extraction and purification of the NGF from the engineered plant.

Herein is hence described the realisation of suitable expression vectors for the production of recombinant human pre-pro-NGF or of recombinant human NGF for the transformation of plant species in transient way and in stable way; an optimized method for carrying out the transient plant transformation mediated by the micro-organism Agrobacterium Tumefaciens; methods for the stable transformation of plants, tissues, or plant cells and the selection of monoinsertional lines that express the Nerve Growth Factor in the form of pre-pro-protein and methods for the purification of the rh pre-pro-NGF protein and of rh NGF produced by transiently or stable transformed plants.

Objects of the present invention are:
I—A dicotyledon plant not grown with feeding intent stably transformed with an expression vector comprising , under the control of a strong constitutive promoter, an expression cassette of nucleic acid comprising: a 5′ UTR sequence, a nucleotide sequence coding for a leader vegetal sequence, a cDNA sequence coding for the human pre-pro-NGF, a nucleotide sequence coding for a sequence that mediates the entry of the protein to which it is bound in the endoplasmic reticulum and a 3′UTR sequence, operatively linked from 5′ to 3′; said plant being capable to constitutively express the human recombinant pre-pro-NGF;
II—the plant described above wherein said cassette further comprises a sequence coding for a purification tag, positioned between said nucleotide sequence coding for a sequence that mediates the entry of the protein to which it is bound in the endoplasmic reticulum and the said 3′ UTR sequence;
III—the plant as described in any one of the preceding points, wherein said cassette further includes, downstream the sequence coding for the pre-pro-NGF, a nucleotide sequence coding for a site of recognition of a protease:
IV—the plant as described in any one of the preceding points, wherein said plant is selected in the group comprising the Dicotyledons with large leaves;
V—parts of the plant as described in any one of the preceding points such as leaves, flowers, shaft, roots, seeds, calluses and protoplasts;
VI—an optimized method for the transient transformation of plants wherein:
a. a growth medium is inoculated with at least one colony of Agrobacterium Tumefaciens transformed with an expression vector capable of directing the in plant expression of a recombinant protein of interest and the cells of the mentioned Agrobacterium Tumefaciens are collected through centrifugation and elimination of the supernatant when the final OD₆₀₀ of growth is of about 1.8-2.0;
b. said cells are suspended in a suitable buffer solution and the said cells are incubated until an OD of growth at 600 nm of about 1.8-2.0 is reached and said cells are collected into a pellet by centrifugation and the supernatant is discarded;
c. said pellet is re-suspended in a suitable infiltration buffer in a volume so that the final OD600 is approximately 1.8-2.0; the infiltration solution thus obtained is inserted in a syringe without needle and a pressure is applied against the of the leaf lamella with said syringe containing said final buffer solution in order to transform in a transient way the portion of the leaf in which the said solution spreads,
this method allowing to obtain an expression of recombinant protein equal to up to 5 times higher than the one obtained with methods described in literature that contemplate the use of a concentration of transformed Agrobacterium having OD₆₀₀ equal to 1 and buffer solutions as showed below;
VII—a method for the stable transformation of dicotyledon plants including the following steps:

Agrobacterium Tumefaciens cells are transformed with an expression vector comprising, under the control of a strong constitutive promoter, an expression cassette of nucleic acid comprising: a 5′UTR sequence, a nucleotide coding sequence, a plant leader sequence, a cDNA sequence coding for human pre-pro-NGF, a nucleotide sequence coding for a sequence that mediates the entry of the protein to which it is bound in the endoplasmic reticulum and a 3′UTR sequence, operatively linked from 5′ to 3′;
leaf discs of said dicotyledon plant are transformed with a solution of said cells of Agrobacterium Tumefaciens;
calluses formation is induced from said leaf discs;
the calluses thus obtained are selected for the presence of said vector;
the selected calluses are used to regenerate full plants that are hemizygous for the cDNA coding for human pre-pro-NGF;
the plants thus obtained are self-fecundated up to the third generation in order to obtain stable transformed plants expressing in a constitutive way the recombinant human pre-pro-NGF;
VIII—the method as described above wherein said cassette further comprises a sequence coding for a tag for the purification of recombinant human pre-pro-NGF localized between the mentioned nucleotide sequence coding for a sequence that mediates the entry of the protein to which it is bound in the endoplasmic reticulum and said 3′ UTR sequence;
IX—the method as described in any one of the previous points, wherein said cassette comprises also, immediately downstream the sequence coding for pre-pro-NGF, a nucleotide sequence coding for a site of recognition of a protease:
X—a method for the purification of rh pre-pro-NGF recombinant from transformed dicotyledon plants expressing said protein comprising the following steps:
   total soluble proteins are extracted through grinding in liquid nitrogen, suspension in a suitable buffer, sonication, centrifugation and recovery of the supernatant solution;
   the solution thus obtained is filtrated using a centrifugal filter (such as Amicon centricon, by way of example)having a cut-off of about 10 Kda allowing the concentration of soluble proteins and the elution of human recombinant pre-pro-NGF on the basis of its molecular weight;
   the thus obtained filtrate is further purified by anion exchange chromatography (commonly used and suitable anion exchange resins are Q-resin, a Quaternary amine; and DEAE res n, DiEthylAminoEthane);
   the thus obtained flow throughs are further loaded charged on cation exchange chromatography (commonly used and suitable cation exchange resins are S-resin, sulfate derivatives; and CM resins, carboxylate derived ions SP-Sepharose);
   the resulting solution is then purified by metal affinity chromatography on a resin matrix functionalized with the nickel metal;
XI—the method as described above wherein said plants or portions thereof are transformed with a vector comprising a sequence coding for a suitable protease recognition site at the 3′ of a sequence coding the cDNA of the recombinant human pre-pro-NGF further comprising the following steps:
   the protein obtained through metal affinity chromatography is incubated with said protease,
   the protein thus obtained is purified by affinity chromatography in a matrix with a resin functionalized with Nickel; in this case the recombinant protein has no affinity with the nickel matrix and hence it is the first to flow (flow through);
XII—a method for the purification of rh pre-pro- NGF from dicotyledon transformed plants or portions thereof expressing said protein comprising the following steps:
   total soluble proteins are extracted through grinding in liquid nitrogen, suspension in a suitable buffer, sonication, centrifugation and recovery of the supernatant solution;
   the solution thus obtained is filtered using a centrifugal filter (such as Amicon centricon or equivalent filters) having a cut-off of about 10 Kda;
   the filtrate thus obtained is further purified by anion exchange chromatography (Commonly used and suitable anion exchange resins are Q-resin, a Quaternary amine; and DEAF resin, DiEthylAminoEthane);
   the flow troughs containing the recombinant human pre-pro-NGF thus obtained are loaded on a cation exchange chromatography (commonly used and suitable cation exchange resins are S-resin, sulfate derivatives; and CM resins, carboxy.iate derived ions SP-Sepharose); for which the recombinant human pre-pro-NGF has a good affinity;
XIII—the method described above wherein said plants are transformed with a vector comprising a sequence coding for the recognition site of a suitable protease at the 3′ of a sequence coding for the cDNA of human pre-pro-NGF further comprising the following steps:
   the protein obtained through ion exchange chromatography is treated by incubation with this protease,
   the protein is purified after this incubation through affinity chromatography with a resin functionalized with Nickel as above described. Alternatively, incubation with the protease may be carried out before the chromatography with a resin functionalized with Nickel described above thus avoiding one chromatographic step.
      In all the purification methods above described it is possible to add a further step to obtain rhNGF, this step foresees the incubation of rh pre-pro NGF protein obtained with each method with a protease selected in the group comprising matrix metal-protease, furine protease, plasmin protease.
      The plant system herein disclosed has the advantage of being an eukaryotic system hence of being compatible with the human system in order to allow a correct production of recombinant human NGF protein (both in the pre-pro NGF form and in the mature protein form), but is also sufficiently far from the human system to avoid the problem of cross-infections, in fact, is well known that plant pathogens do not infect humans.
      In order to render the pre-proNGF and NGF production easy, low cost and of fast industrial scaling up, the system herein proposed bases on the construction of plant expression vectors that renders all the steps above easy and immediate.

Summarising, among the possible advantages of the present invention with respect to disadvantages of state of the art are, to provide in a single solution:
obtaining a protein with a correct folding thanks to the use of an eukaryotic system capable to perform post transcriptional and post traditional modification similar to those performed by the human system;
using a safe system because plant pathogens are not harmful for humans, which implies a low contamination risk and low cost sterility conditions;
an easy recovery of recombinant pre-proNGF and NGF protein thanks to the presence in the plant protein expression vector of sequences that can simplify and maximize the yields of protein separation from the other proteins present in the plant.
an easy industrial implementation thanks to the use purification techniques and protocols easily exportable in a large scale production that does not require the realisation of particular plants as methods derived from homeopathic industry and/or food industry can be used.

### DETAILED DESCRIPTION OF THE FIGURES AND OF THE SEQUENCES

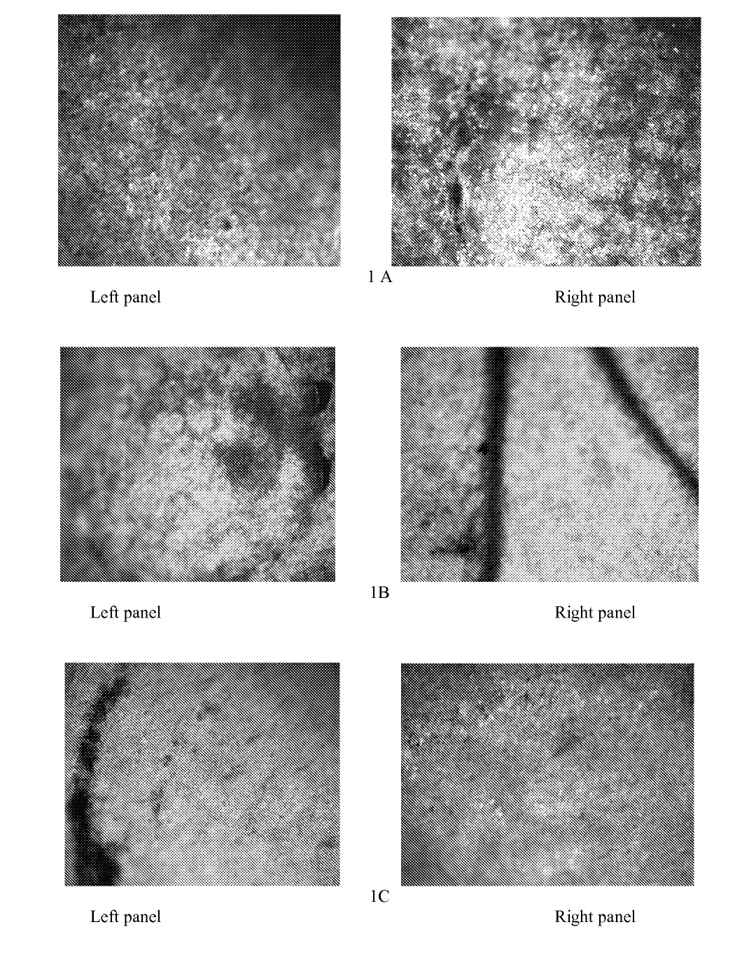

FIG. 1: Comparison between standard infiltration protocol by Agrobacterium Tumefaciens and modified protocol.

Leaf tissue by Nicotiana benthamiana observed using a florescence microscope:

The comparison between the two protocols was performed by infiltrating an equal quantity of infiltration buffer, containing Agrobacterium Tumefaciens transformed with a GFP expression vector, in leafs belonging to the same plant. This assay removes all doubts about the less or more buffer quantity inoculated in a plant rather than in another plant and doubts about transformed plant physiologic conditions.

In the left column are reported the photos of the standard protocol, in the right column the ones of the protocol of the invention.

PANEL (A), three days post-infiltration without p19; the modifications made of the standard protocol have as consequence a higher GFP expression on plants infiltrated without p19 (gene silencing suppressor) after three days post transient infiltration.

Moreover the right photo shows how the modifications of the syringe used for the infiltration makes the GFP signal more equally distributed inside the leaf tissue.

PANEL (B) seven days post infiltration with p19;

The changes made on the standard protocol have as consequence a higher GFP expression on infiltrated plants in the presence of p19 (gene silencing suppressor) after seven days post transient infiltration. Also in this case the right signal, related to the modified protocol with p19 and performed with modified syringe, assure higher expression uniformity inside infiltrated tissue.

The invention protocol assures expression uniformity in all infiltrated cells.

PANEL (C) seven days post-infiltration without p19.

In these images it is highlighted how, after seven days post infiltration without p19, using the standard protocol the florescence signal is absent; while with the modified protocol the florescence signal still persist even if in low.

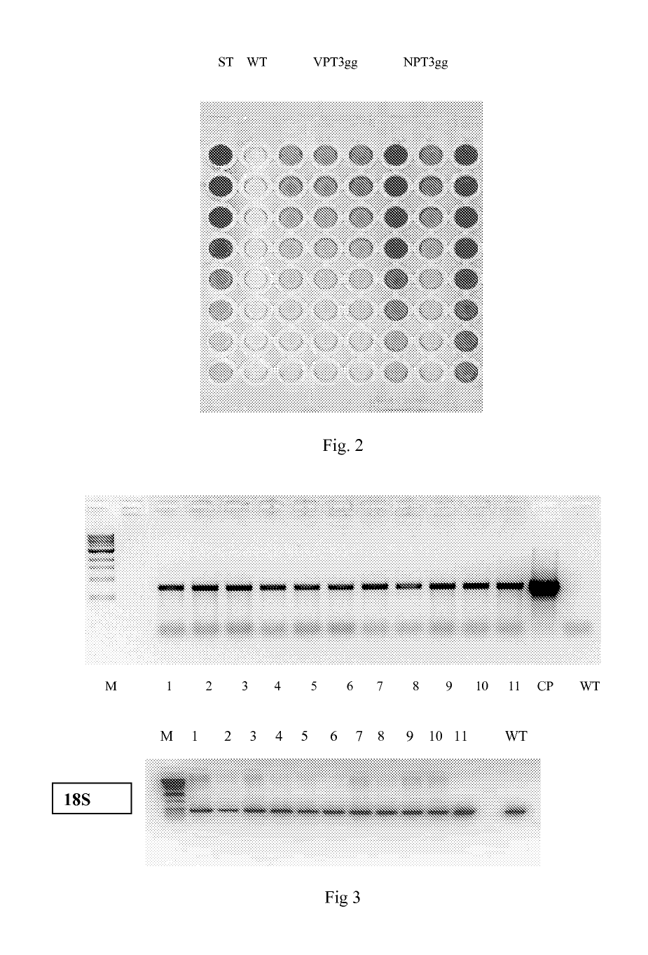

FIG. 2. Elisa test on transient transformed plants with NGF-vec-OLIGO vector.

Comparison between standard Agrobacterium tumefaciens infiltration protocol and modified protocol.

St: NGF standard
WT: not transformed plants
VPT 3gg: standard transient transformation protocol after 3 days at different dilutions.
NPT 3gg: news transient transformation protocol after 3 days at different dilutions.

The concentrations are of about 5 μg/Kg of fresh leaf using the invention protocol VS 1 μg/Kg of fresh leaf using standard protocol.

FIG. 3: RT-PCR on cDNA derived from transient transformed plants and stable transformed plants(GAPIone) using primers on gene coding for pre-proNGF:

M: molecular weight marker 1 Kb.
1-2-3-4-5: cDNA derived from transient transformed plants
6-7-8-9-10-11: cDNA derived from stable transformed plants GALPIone
CP: positive control-plasmid DNA of NGF-vec-OLIGO
WT: wild type plants
18S: house kipping gene of Nicotiana benthamiana used as quality RNA control.

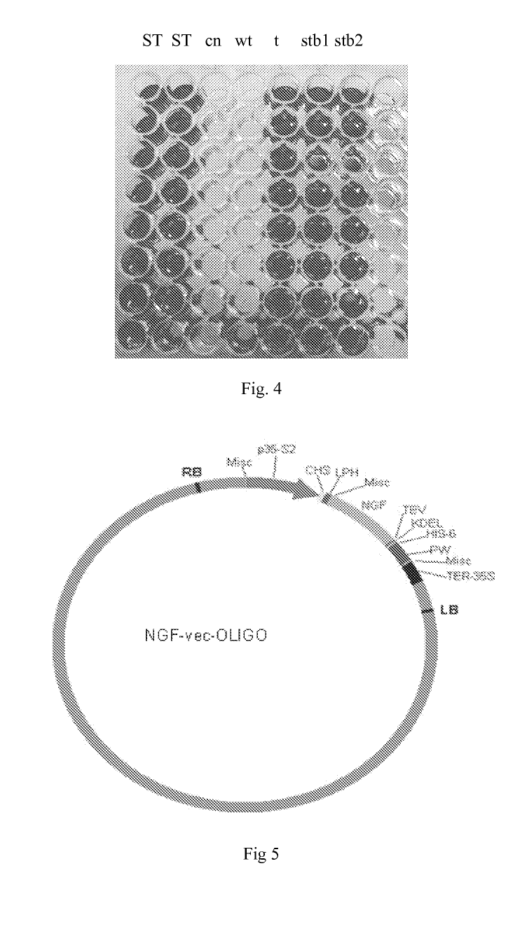

FIG. 4: Elisa Test on stable transformed plants with NGF-vec-OLIGO vector GALPIone line plants transiently transformed with the same vector.

ST=standard NGF.
ST=standard NGF.
Cn=negative control.
Wt=not transformed plant.
T=Transiently transformed plant with NGF-vec-OLIGO vector, samples repeated in the column.
Stb1=Stable transformed plant with NGF-vec-OLIGO vector GALPIone 1, samples repeated in the column.
Stb2=Stable transformed plant with NGF-vec-OLIGO vector GALPIone 2, samples repeated in the column.

The obtained yields are approximately from 3 to 5 μg of recombinant protein per kg of fresh material both for the stable GALPIone transformation and the transient transformation.

The Elisa test highlights that also the stably transformed plants, GALPIone, produce pre-pro-NGF and the production yield between transient and stable shows no differences.

FIG. 5: Expression vector NGF-vec-OLIGO

P35S=constitutive promoter double 35S of Cauliflower Mosaic Virus (CaMV)
CHS=5′ UTR sequence deriving from chalcone synthase
LPH=leader peptide
NGF=cDNA coding for the pre-pro-NGF form
TEV=recognition site for the Tobacco Etch Virus protease
KDEL=sequence that mediates the entry of the protein in the endoplasmic reticulum
6×-His tag=sequence of six histidines
PW=3′ UTR region of Tobacco Mosaic Virus
TER-35S=35S terminator of Cauliflower Mosaic Virus.

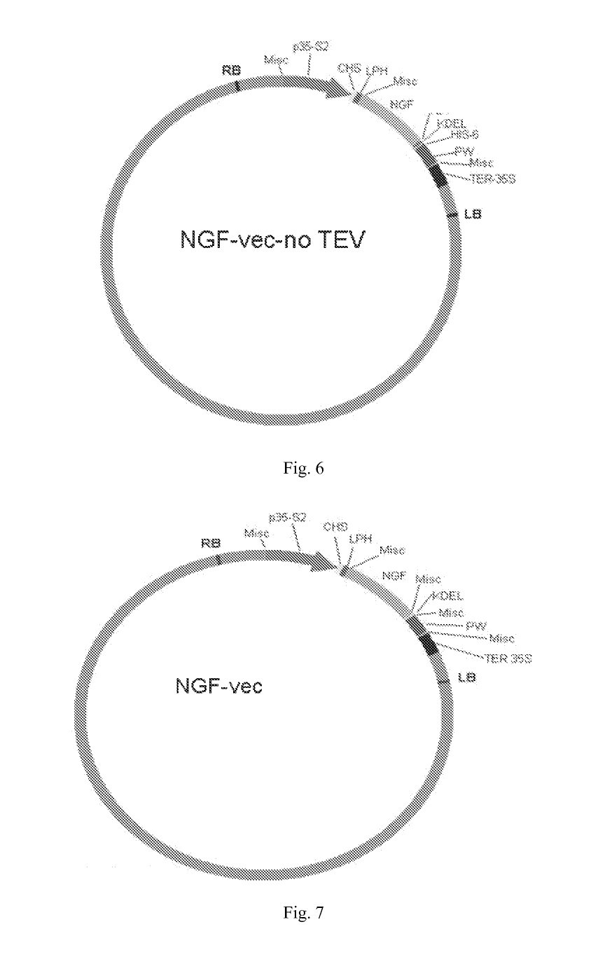

FIG. 6: Expression vector NGF-vec-no TEV

P35S=constitutive promoter double 35S of Cauliflower Mosaic Virus (CaMV)
CHS=5′ UTR sequence deriving from chalcone synthase
LPH=leader peptide
NGF=cDNA coding for the pre-pro-NGF form
KDEL=sequence that mediates the entry of the protein in the endoplasmic reticulum
6×-His tag=sequence of six histidines
PW=3′ UTR region of Tobacco Mosaic Virus
TER-35S=35S terminator of Cauliflower Mosaic Virus.

FIG. 7: Expression vector NGF-vec

P35S =constitutive promoter double 35S of Cauliflower Mosaic Virus (CaMV)
CHS=5′ UTR sequence deriving from chalcone synthase
LPH=leader peptide
NGF=cDNA coding for the pre-pro-NGF form
KDEL=sequence that mediates the entry of the protein in the endoplasmic reticulum
PW=3′ UTR region of Tobacco Mosaic Virus
TER-35S=35S terminator of Cauliflower Mosaic Virus.

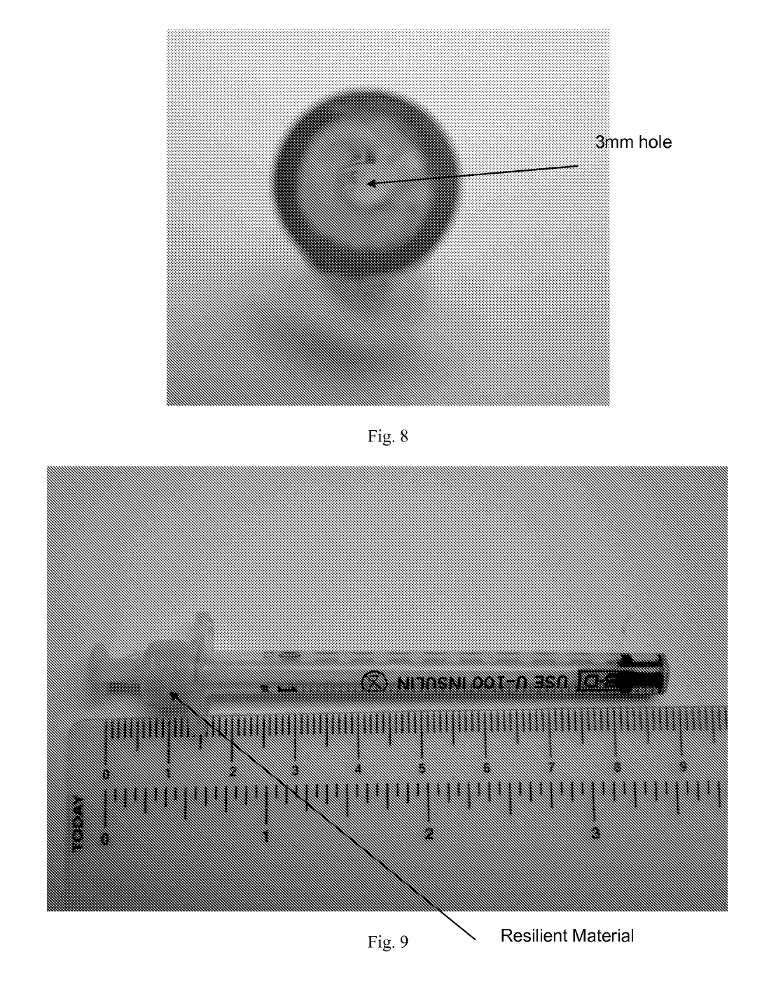

FIGS. 8 and 9 represent the syringe of the invention in detail.

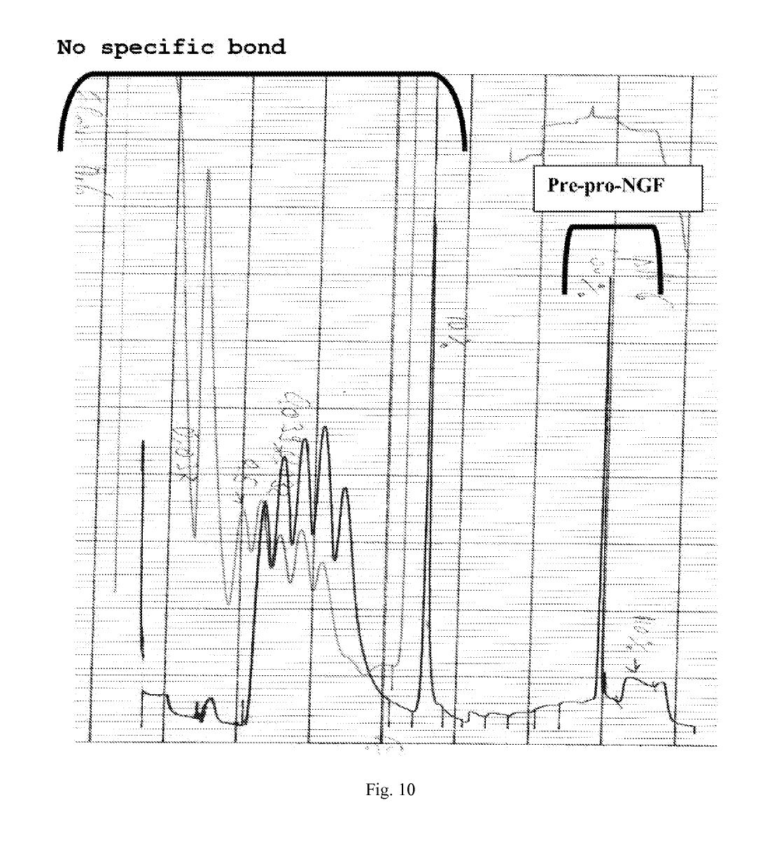

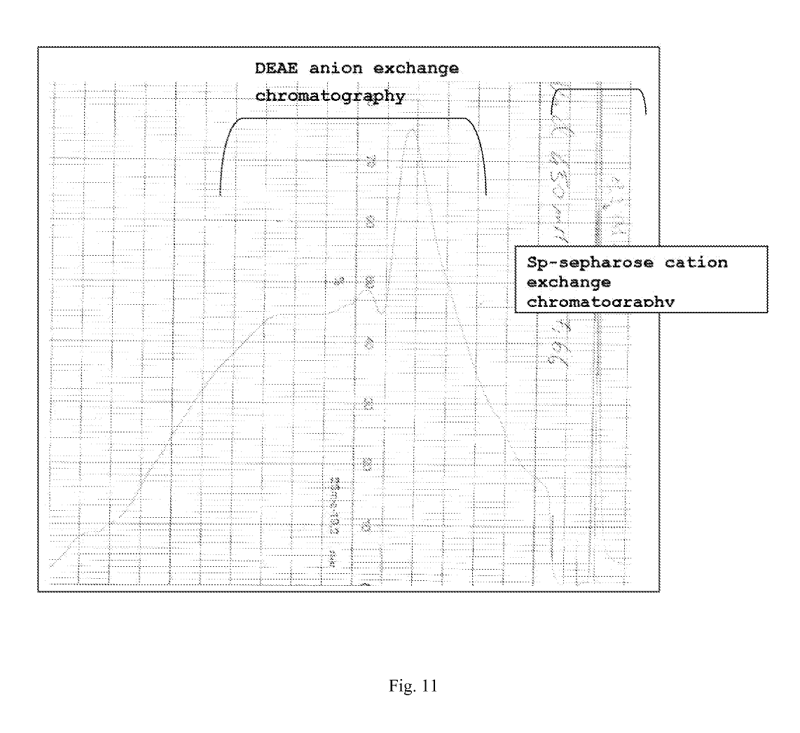

FIG. 10 rh pre-pro-NGF purification. The figure shows the metal affinity chromatograpy with a matrix functionalised with nikel (Ni-nta) registered by and UV record at 280 nm The peaks indicated as “No specific bond” indicate the proteins without poly His tails and are eluted first, the peak indicated as “pre-pro NGF” relates to the pre-pro-NGF recombinant protein having poly His tails and hence being eluted last, with a buffer comprising Pi 75 mM pH 7.5, NaCl 450 mM+50 mM imidazole, ph 6.

FIG. 11 represents a chromatogram of the pre-pro- NGF purification by anion exhange chromatography followed by cation exchange chromatography. The figure shows the chromatogram of the purification phases of the rh pre-pro Ngf by a first anion exchange chromatography on DEAE. The chromatography development has been followed by an UV record set at 280 nm wavelength showing the presence of a peak, herein indicated as L′immagine mostra it cromatogramma relativo alle fasi di purificazione della proteina ricombinante pre-pro-NGF per mezzo di una prima cromatografia a scambio anionico con resina DEAE. “DEAE anion exchange chromatography” comprising the rh pre-pro-NGF protein having low affinity for the matrix used and being hence directly eluted as flow-through. The figure shows also the chromatogram related to the cation exchange chromatography that follows wherein the flow through obtained by the DEAE column is directly loaded on a colum functionalised with SP-sepharose, which is a matrix for which the pre-pre-NGF has a very good affinity. The development of the chromatography has been recorded by an UV record set at 280 nm wavelength and the peak indicated as “Sp-sepharose cation exchange chromatography” relates to the fraction eluted with the Pi 75 mM pH 7.5, NaCl 450 mM buffer containing the rh pre-pro-NGF.

Seq Id 1 represents the known nucleotide sequence of the human pre-pro-NGF.

Seq Id 2 represents the known amino acid sequence of the human pre-pro-NGF. Seq Id 3 represents the pre- sequence of the known amino acid sequence of the human pre-pro-NGF localized in position 1-18 of Seq ID 2.

Seq Id 4 represents the -pro- sequence of the known amino acid sequence of the human pre-pro-NGF localized in position 19-121 of Seq ID 2.

Seq Id 5 represents an expression cassette in accordance with the description, herein called NGF-VEC-OLIGO, comprising:

### Position:

| | |
|---|---|
| 19-720 | 35S² promoter |
| 721-774 | CHS |
| 775-824 | LPH |
| 825-837 | Misc. derived from binary vector |
| 838-1554 | CDS gene NGF |
| 1560-1581 | site TEV protease |
| 1587-1599 | KDEL |
| 1600-1618 | Histidine tail (His₆) |
| 1625-1828 | PW |
| 1829-1847 | Misc. derived from binary vector |
| 1847-2073 | 35S terminator |

Seq Id 6 represents an expression cassette in accordance with the description, herein called NGF-VEC, comprising:

### Position:

| | |
|---|---|
| 19-720 | 35S² promoter |
| 721-774 | CHS |
| 775-824 | LPH |
| 825-837 | Misc. derived from binary vector |
| 838-1554 | CDS gene NGF |
| 1555-1595 | Misc. derived from binary vector |
| 1596-1607 | KDEL |
| 1608-1619 | Misc. derived from binary vector |
| 1620-1823 | PW |
| 1824-1843 | Misc. derived from binary vector |
| 1844-2070 | 35S terminator |

Seq Id 7 represents an expression cassette in accordance with the description, herein called NGF-VEC-OLIGO no TEV, comprising:

Position:

| | |
|---|---|
| 19-720 | 35S² promoter |
| 721-774 | CHS |
| 775-824 | LPH |
| 825-837 | Misc. derived from binary vector |
| 838-1554 | CDS gene NGF |
| 1560-1572 | KDEL |
| 1573-1591 | Histidine tail (His₆) |
| 1598-1801 | PW |
| 1802-1820 | Misc. derived from binary vector |
| 1821-2047 | 35S terminator |

Seq Id 8 represents the oligonucleotide used for the insertion of the sequences: TEV-KDEL-6×His-Tag

Seq Ids 9-13 represent the primers used for the construction of the expression cassettes.

Seq Id 14 represents the amino acid sequence of the recognition site for the protease of Tobacco Etch Virus, TEV.

### DETAILED DESCRIPTION OF INVENTION

Mature NGF herein means the β-NGF without the signal sequence “pre” and the propeptide sequence “pro”.

Pre-pro-NGF herein means β-NGF with the signal sequence “pre” and the propeptide sequence “pro”. Stable Transformation herein means an approach of transformation in which the gene of interest, in this case coding for pre-pro-NGF and Mature form of NGF, is stably integrated inside the plant genome, is present in all the cells and is inherited by the following generations.

Transient transformation herein means an approach of transformation limited in time and restricted to a limited number of somatic cells, wherein the gene of interest coding for the pre-proNGF and for mature NGF is not stably integrated in the plant genome and is not inherited by following generations.

Infiltration herein means the step that is performed in transient transformation method in which a culture of Agrobacterium Tumefaciens transformed with the expression vector of interest, is spread through leaf lamella cells using a syringe and applying a low pressure on the leaf lamella so that the transformed bacterium penetrates inside the somatic cells to which it is contacted allowing the transient expression of recombinant protein coded by the expression vector of interest.

Recombinant pre-proNGF or recombinant NGF herein means the pre-proNGF or Mature NGF obtained by organism different from the organism of origin.

hNGF herein means human NGF, rhNGF herein means the recombinant human NGF protein, the same is for pre- proNGF.

Plant tissue herein means a set of plant cells specialized to make particular functions.

Plant molecular Farming herein means the use of plants as bio-reactors for the production of recombinant bio-molecules of pharmaceutical and/or industrial interest.

Bio-reactor herein means a device for the growth of organism such as bacteria, yeast, used in biotechnology for the production of compounds such as pharmaceuticals, antibodies, vaccines, or for the bio-conversion of organic wastes.

Mono-insertion line herein means a plant that has inside of its genome a single copy of an exogenous DNA coding for an exogenous protein.

Gene cassette herein means the information sequence carried inside the portion of an exogenous DNA inserted in an expression vector to be inserted or inserted inside a plant conferring to said vector the capability of inducing the expression the protein of interest upon introduction into the plant.

Constitutive promoter herein means a nucleotide sequence capable to promote the transcription of sequences contiguous to this sequence, regardless of the presence or not of particular external stimulations or physiologic conditions, in an undifferentiated way in all plant tissues.

Folding herein means the process by which the proteins reach their three dimensional structure. The folding takes place during the protein synthesis and the end of protein synthesis. Only after the folding is terminated the proteins can gain their physiologic function.

UTR sequence herein means untranslated region i.e. non coding region. The untranslated regions are RNA sections localized before the start codon and after the stop codon, that are not translated and that are named 5′ UTR and 3′ UTR. Several roles have been ascribed to the non coding regions in the gene expression, including the mRNA stabilization, the mRNA localization and the translational efficiency. The mRNA stability can be mediated by 5′ UTR and by 3′ UTR, because of changeable affinity for some enzymes capable of degrading the DNA, the ribonucleases, that may promote or inhibit the stability of the RNA molecule. More an mRNA is stable and more proteins can be produced from that transcription.

Operatively linked is herein referred to the association of two o more fragments of nucleic acids in a way that the function of one is linked to the function of the other. By way of example, a promoter will be operatively linked to a sequence to be transcribed, when it is capable to regulate the expression of said sequence (i.e. the coding sequence is operatively linked to the promoter when it is under the control of said promoter). The coding sequences can be operatively linked to regulation sequences in sense or anti-sense orientation.

The term “OD600” in this description, means the OD of a transformed Agrobacterium growth culture at 600 nm.

The word “Tag” in this description, means any one of the mans known in the state of the art to purify a protein with a method that envisages a modification of the protein with an epitope which allows the purification system used to recognize in a selective way the protein, system of this kind are the His-tag system, the FLAG system and other systems listed afterwards.

The word “plant grown for alimentary purposes” is used in this description to indicate plant crops that are grown as food for humans, from the definition of “plant grown for alimentary purposes” all not poisonous plants, and for that theoretically edible ,that are not grown as food for human use are not included. Hence, the definition “plant not grown for alimentary purposes” comprises all those plants that are not grown or used for feeding humans. Plants used for animal feeding might be included but may be excluded as well from said definition. The mere fact that a plant is edible, as long as it is not used for human feeding, does not exclude said plant from the definition “plant not grown for alimentary purposes”.

The word “callus” indicates a plant undifferentiated tissue.

The word protoplast indicates a plant cell to which the cellular wall has been removed by an enzyme such as cellulase and pectinase. Protoplasts can be isolated from different plant tissues such as: leafs, stem, root, flowers, anther and pollen.

The method dicotyledon plants transformation according to the description foresees the construction of expression vectors comprising a gene cassette under the control of a promoter wherein said cassette has at least the following components, ordered from 5′ to 3′, operatively linked:
a 5′ UTR sequence for RNA stabilisation
a nucleotide sequence coding for a plant leader peptide
a cDNA sequence coding for the human pre-proNGF
a nucleotide sequence coding for an amino-acid sequence capable of mediating the entry and the retention of the expressed protein inside the endoplasmic reticulum
a 3′ UTR sequence.

The above described cassette allows the expression, in plants transformed with the vector comprising the cassette as described, of recombinant human pre-proNGF with a correct folding.

The cassette will be positioned in a vector, in a region comprised between the promoter that controls the cassette and a site of transcription termination that shall be present inside the expression vector.

Advantageously, the cassette may also comprise a tag to easy the protein purification, this tag will be positioned before the 3′ UTR region and after the sequence mediating the entrance and the retention of the expressed protein inside the endoplasmic reticulum.

This tag can be any kid of tag known in the art such as, but not limited to these, the his-tag (ex: 6×his tag), or other well known as GTS (glutathione s-transferase), ZZ (immunoglobulin), Strep-Tag (streptavidine), MBP(amylose) and similar.

Moreover, the cassette may advantageously contain a recognition site for a suitable protease allowing the easy removal of the sequences located at the C-terminal if the recombinant protein.

The 5′ UTR sequence according to the description may be any 5′ UTR sequence suitable for the stabilization of the protein of interest, among those, the CHS (5′ UTR chalcone synthase sequence).

The leader peptide is a leader peptide of plant origin hence optimal for the expression, in plant, of proteins, even heterologous ones. An example of such leader sequence optimized as described can be seen in Seq ID 5, in Seq ID 6 and in Seq ID 7 in position 775-824 of each of the above sequences.

The human pre-proNGF cDNA sequence is kwon in literature and is anyhow reported in Seq ID 1, the sequence “pre” in reported in Seq ID 3, the sequence “pro” is reported in Seq ID 4. From the sequences in Seq ID 1, 3 and 4 the skilled person can easily deduce the hNGF sequence that will hence be equivalent to the aa from 122 to 241 of Seq ID 1 or 2, and to the nucleotides 3364-723 of Seq ID 1.

Regarding the sequence for the binding to the endoplasmic reticulum, it is known that the majority of the proteins inside the endoplasmic reticulum are retained therein trough an amino-acid motif responsible of the keeping inside. This motif is composed of four amino-acids at the end of the protein, the most common sequence for retention is KDEL described as DNA sequence in position 1587-1599 of Seq ID 5, 1596-1607 of Seq ID 6 and in position 1560-1572 of Seq ID 7 (lys-asp-glu-leu), however, other amino-acid sequences capable of retaining the protein inside the endoplasmic reticulum are obviously suitable for carrying out the invention.

Concerning the 3′ UTR region, any suitable 3′ UTR known to the skilled person can be used, by way of example, the PW 3′ UTR of mosaic tobacco virus.

To easy the protein purification a tag sequence as described above may be used.

The protease recognition site according to the description may be, by way of example, the TEV (tobacco etch virus) described as nucleotide sequence from which the amino-acid sequence of Seq ID 14 is obviously derivable, in position 1560-1581 of Seq ID 5.

Other suitable proteases known in literature, the recognition site of which may hence be inserted in the expression cassette as above described, can be enterokinase, trombin, Xa factor and similar; but the description is not limited to these proteases.

The expression cassette as described shall be posed under the control of a promoter, when the expression of protein in all or most of all plant tissues it may be convenient to use a constitutive or semi-constitutive promoter. Numerous constitutive promoters suitable for in plant expression are known in literature such as, by way of example, the 35S CMV enhanced promoter (cauliflower mosaic Virus), rice actin promoter, Corn Ubiquitin promoter, NOS (Nopaline synthase) promoter, OCS (octopine synthase) promoter. It is clear that the plants with highest production will be those under the control of strong constitutive or semi-constitutive promoter such as, by way of example, the enhanced 35S CMV promoter (Cauliflower Mosaic Virus)(enhanced 35S CMV), and the others reported above.

According to the present description, some embodiments for carrying out vectors suitable for dicotyledonous plants transformation for the expression recombinant human pre-proNGF are:

Vector 1, herein named NGF-vec-Oligo

In the NGF-vec-Oligo vector (FIG. 5) the cDNA coding for the pre-pro-NGF is inserted in a gene cassette containing:

CHS'5′ UTR sequence derived from chalcone synthase having an RNA stabilisation function.

LPH=leader peptide having optimized codons for translation in plant.

NGF=cDNA coding for the pre-pro-NGF form.

TEV=tobacco ecth virus site (Seq Id 14) recognition site useful for the removal of the C-terminal sequences on the recombinant protein.

KDEL=sequence mediating the entrance and the retention of protein inside the endoplasmic reticulum.

6×-His tag=six-histidine sequence useful for the purification of the protein by affinity chromatography.

PW=3′ UTR region of tobacco mosaic virus.

This gene cassette is under the control of the double 35S (enhanced promoter 35S of cauliflower mosaic virus) promoter and has as the 35S transcription terminator of cauliflower mosaic virus.

This vector is designed for the producing of a chimeric protein made of the “pre” signal sequence, the pro-peptide sequence “pro”, the NGF mature form, the sequence for the proteolytic cut by TEV protease, of the KDEL sequence for the retention of the protein in endoplasmic reticulum and of the histidine tail for the protein purification by affinity chromatography.

By the proteolytic cut with TEV a chimeric protein with only the sequences “pre”, “pro” and NGF is obtained.

Vector 2, herein named NGF-vec-NO-TEV

In the NGF-vec-NO-TEV vector (FIG. 6) the cDNA coding for the pre-pro-NGF is inserted in a gene cassette containing:

CHS=5′ UTR sequence derived from chalcone synthase for the RNA stabilisation.

LPH=leader peptide with codons optimized for translation in plant

NGF=cDNA coding for the pre-pro-NGF form.

KDEL=sequence mediating the protein entrance and the retention inside the endoplasmic reticulum.

6×-His Tag=six-histidines sequence for the protein purification by affinity chromatography.

PW=3′ UTR region of tobacco mosaic virus.

This gene cassette is under the control of the double 35S (enhanced promoter 35S of cauliflower mosaic virus) promoter and has the 35S transcription terminator of cauliflower mosaic virus.

This vector is designed for the production of a chimeric protein made of the “pre” signal sequence, the pro-peptide sequence “pro”, the NGF mature form, the KDEL sequence mediating the protein entrance and retention in the endoplasmic reticulum and the histidine tail for the protein purification by affinity chromatography.

Vector 3, nominated NGF-vec

In the NGF-vec vector (FIG. 7) the cDNA coding for the pre-pro-NGF is inserted in a gene cassette containing:

CHS=5′ UTR sequence derived from chalcone synthase for stabilize the RNA.

LPH=leader peptide with optimized codon for translation in plant

NGF=cDNA coding for the pre-pro-NGF form.

KDEL=sequence for the retention of protein inside the endoplasmic reticulum.

PW=3′ UTR region of tobacco mosaic virus.

This gene cassette is under double 35S (enhanced promoter 35S of cauliflower mosaic virus) promoter and under control of 35S transcription terminator of cauliflower mosaic virus. This gene cassette is under control of the double 35S (enhanced promoter 35S of cauliflower mosaic virus) promoter and has the 35S transcription terminator of cauliflower mosaic virus.

This vector designed for the production of a chimeric protein constituted of the “pre” signal sequence, the pro-peptide sequence “pro”, the NGF mature form, and of the KDEL sequence for the internalisation and retention of the protein in the endoplasmic reticulum.

The described vectors have been used to transform, both transiently and stably dicotyledonous plants and hence to constitutively induce the synthesis of human recombinant pre-pro NGF in said plants.

According to the present description, the plant transformed for the production of rhNGF, or human recombinant pre-proNGF, is a dicotyledonous plant. The choice of the ideal dicotyledonous is bound to the GMO plants problems in general, and accounts also for the farming problems. This means that it is intended to avoid the risk of contamination of plants used as foods by GMO material (contamination is the sense of propagation of heterologous traits in plants for food use due to the pollination of the same plants by the plants described). It is kwon that the majority of plants cultivated for human feeding are represented monocotyledonous such as rice, wheat, soybean etc. Consequently, the dicotyledonous plants suitable for the carrying out of the description are those dicotyledonous that are not used for human feeding and, optionally, even not for animal feeding. The dicotyledonous plants used for alimentary use are excluded from the carrying out of the present description, not because the method is technically unachievable also in those plants.

The selection of the present description within the dicotyledonous plants is mare, mainly; in order to avoid “contaminations” of edible plants that are cultivated for alimentary scopes. Among the selected plants there are, by way of example, Nicotiana tabacum, Nicotiana benthamiana and more in general all inedible dicotyledonous edible plants with large leaf, in particular the plants of Solanacee family, that comprises about 1400 species, are preferred.

Another advantageous characteristic of the plant described may be the easy farming of the same and even more advantageous may be the use of plants in which the transformation protocols mediated by Agrobacterium are known as effective.

The plant according to the present description is hence a dicotyledonous plant that is not grown for alimentary purposes, stably transformed by an expression vector comprising, under the control of a strong constitutive promoter, a nucleic acid expression cassette comprising: a 5′ UTR sequence, a nucleic acid sequence coding for a plant leader sequence, a cDNA sequence coding for the human pre-proNGF, a nucleic acid sequence coding for a sequence mediating the entrance and the retention in the endoplasmic reticulum, a 3′ UTR sequence, operatively linked from 5′ to 3′, said plant being apt to express the recombinant human pre-proNGF. Advantageously, the cassette may also comprise a sequence coding for a purification tag positioned between the nucleotide sequence coding for the sequence mediating the entry and the retention of the protein to which it is bound in the endoplasmic reticulum and the 3′ UTR sequence. The tag according to the description may be any tag for protein purification well kwon to the skilled person such as, by way of example, but not limited to these, the his-tag (6×his tag) or others known to the skilled person such as GST (glutathione s-transferase), ZZ (immunoglobulin), STREP-Tag (streptavidin), MBP (amylose) and similar.

Said tag, will have the advantage of facilitating the purification of the protein to which it is bound, i.e. the human recombinant pre-proNGF produced by the plant. Depending on the tag selected, the skilled person will know which step to carry out for the separation of recombinant pre -pro-NGF bound thereto following standard protocols for the selected tag. By way of example, as known to the skilled person, for his-tags imidazole will be used, for strep-tag imminobiotin will be used, for ZZ tag the purification will be carried out depending on the pH, for MBP maltose will be used and for GST reducing compounds will be used.

Moreover, the expression cassette may also comprise, downstream of the sequence coding for pre-pro-NGF, a protease recognition site which will allow the cut of all that is expressed to C-terminal of the pre-proNGF produced by the plant of the description. This will hence allow to obtain a protein without different extension at the C-terminal with respect to the native protein. Suitable protease cut sites (recognition sites), may be represented by cutting sites for TEV protease, enterokinase, trombin, factor Xa and similar but the invention is not limited to those.

The embodiments described above allow the production of a fusion protein easy to purify even from tissues wherein the purification toilsome such as the plant tissues.

Usually, prior to perform a stable transformation, it is advantageous to perform a transient transformation, which allows to rapidly verify in the effectiveness of the vector, and hence its capability to induce the expression of the desired protein in a constitutive and effective way.

The stably transformation method of the present description, is a method wherein a vector according to the description is used for plant transformation as described, by the use of an Agrobacterium Tumefaciens solution containing said expression vector. The Agrobacterium mediated transformation by is know to skilled person (e.g. as described by: Patric Gallios and Paulo Marinho plant gene transfer and expression protocols” leaf disk transformation using Agrobacterium Tumefaciens-Expression of Heterologous Genes in Tobacco” Series: Methods in molecular biology, volume:49, Pub. Date: Sep. 27, 1995, Page range: 39-48, DOI: 10.1385/0-89603-321-X:39), it is however herein described in a general way.

Leaf disks of the selected plant are transformed with the said solution. Subsequently, the formation of calluses that will be selected to verify the effectiveness of the vector is induced on a medium containing the anti-biotic for which the vector confers a resistance. The resistant calluses are hence regenerated giving raise to plants hemizygous for the gene coding the protein pre-pro-NGF and NGF protein.

The plants that are hemizygous for the gene of interest are self-pollinated up to the third generation obtaining from these, seeds capable of developing in plants containing the expression cassette for the NGF production and capable to transfer in an irreversible way said cassette to the following generations.

Thanks to the use of a strong constitutive promoter the pre-proNGF is expressed in all plants parts (roots, Shaft, leafs, flower and seeds) and in all plant developing stages (from seed to new seed).

The pre-proNGF production in stably or transiently transformed plants can be analyzed by RT-PCR on cDNA derived from leaf tissue as reported in FIG. 3, or by quantitative ELISA test as reported in FIG. 4. These assays do not require further teachings in the present description being commonly known by the skilled person.

The method can be described as follows:
a method for stably transforming dicotyledonous plants comprising the follow steps:
cells form Agrobacterium Tumefaciens are transformed with an expression vector comprising a nucleic acid expression cassette under the control of a strong constitutive promoter, comprising a 5′UTR sequence, a nucleotide sequence coding for a plant leader sequence, a cDNA sequence coding for human pre-pro-NGF, a nucleotide sequence coding for a sequence that mediates the entry of a protein to which it is bound into the endoplasmic reticule, a 3′ UTR sequence, operatively linked, form 5′ to 3′;
leaf discs from said plant are transformed with a solution comprising said Agrobacterium Tumefaciens cells; formation of calluses from said leaf discs is induced;
the calluses thus obtained are selected for the presence of said vector;
the thus selected calluses are used for regenerating whole plants that are hemizygous for the cDNA coding for the human pre-pro-NGF;
the thus obtained plants are allowed to self fertilise up to the third generation so to generate plants that are stably transformed with said vector that are capable of constitutively expressing human recombinant pre-pro-NGF.

All described above about vectors and plants applies to the above describes methods.

Consequently, vectors with expression cassettes according to one of all the embodiments above described may be used in any of the dicotyledonous plants that are not for alimentary purposes as indicated above.

As indicated before, instead of whole plants, calluses or protoplasts resulting from the transformation methods described below can be used.

The methods described below, when related to calluses or protoplasts, foresees when calluses are to be produced, of the method only up to the callus generation without the regeneration plant, when protoplasts are to be produced, the method foresees isolation of protoplasts from the transformed tissue by any method known to the skilled person.

Protoplasts can be isolated from different plant tissues such as: leafs, stems, roots, flowers, anthers and pollen.

Protoplasts can be stably or transiently transformed by techniques such as:
electroporation, bombardment, Agrobacterium Tumefaciens.

The protoplasts cells most commonly used are the BY-2 cells of Nicotiana tabacum because they can be directly co-cultivated at the presence of Agrobacterium.

The cultures media are the same used for the stable or transient transformation.

Three days after the co-cultivation at the presence of Agrobacterium a transient expression is observed.

Eight weeks after and after several steps in selective media a stable protoplasts transformation is observed.

Starting from protoplasts, is possible to induce calluses formation and than whole plant regeneration.

As before described, it can be advantageous to perform a transient transformation in order to evaluate the effectiveness of the vector to use for the transformation.

Transient transformation consists of infiltration in plant leafs tissues mediated by Agrobacterium Tumefaciens carrying the expression vector of interest. There are several protocols available for said technique.

However, the authors of the present description have optimized the protocols known in literature obtaining a transient transformation method wherein the recombinant protein expression produced by the transiently transformed somatic cells, is up to 5 times higher with respect to the expression obtained using the protocol described in (Tadeusz Wroblensky et. Al-Plant biotechnology journal vol 3, pp 259-273-2005; Jyoti Kapila et.al -Plant science vol.122, pp 101-108-1997).

According to the state of the art, this kind of transformation allows to obtain a recombinant protein production localized only at the infiltration site and time-limited.

To carry out this transformation a 1 ml syringe without needle is used, a pressure is applied on the leaf lamella, so that the buffer solution containing the Agrobacterium transformed with the expression vectors, is spread inside the leaf (infiltration) that will express the protein only in the infiltrated infiltrated tissues for a limited time. This system has the advantage to being fast, of allowing an immediate check of the correct functioning of the expression vectors of avoiding the creation of a genetically modified organism as the inserted trans-gene is not inherited by the following generations.

The preparation of the infiltration mean and of the culturing media is widely described in literature (Tadeusz wrobleski et.al -Plant Biotechnology Journal vol.3, pp 259-273-2005; Jyoti Kapila et al -Plant science vol.122, pp 101-108-1997).

Herein is described an improvement of the agro-infiltration technique by the use of some important modifications of standard protocols and, optionally, of a syringe suitably modified to carry out the infiltration.

These modifications concern the optimization of the growth parameters of Agrobacterium Tumefaciens culture used to infiltrate the lower side of leaf lamella, the buffers used for the transformation and, optionally, also a modification of the syringe used for said infiltration.

According to the descriptions available in literature, the infiltration with Agrobacterium is carried out when the Agrobacterium tumefaciens culture o used for the transformation has the OD 600 equal to 1. In the method of the present description the infiltration is carried out when the OD600 is comprised between about 1.8 and 2.0. The authors have selected this OD 600 range because they have surprisingly discovered, that, using for the infiltration a culture of Agrobacterium Tumefaciens transformed with by expression vector of interest at an OD 600 comprised between about 1.8 and 2.0 a much more effective transient transformation was obtained.

Moreover, the authors of the present description have further optimized the transient transformation method through the modification of the tools used for the infiltration.

The syringe used for agro-infiltration must allow the entry of the solution containing the Agrobacterium transformed with the vector with the gene for the hexogen protein production, in a way that will not necrotise the tissue upon the syringe stand (necrotised tissues will not produce the protein). The syringe must ensure the liquid entry inside the leaf at constant pressure in order to allow the plant cells to adsorb as much solution as possible, obtaining as a result an infiltration of the largest leaf portion possible and a subsequent uniform expression of the recombinant protein.

In order to confer to the infiltration instrument these characteristics some modification has been made on a common 1 mL insulin syringe.

The modifications are illustrated in FIGS. 8 and 9.

In order to render the syringe not dangerous for the leaf tissue, the support for the needle insertion has been removed from a common thereby creating a smooth opening of about 3 mm that does not cause tissues necrosis.

In order to ensure a suitable and almost constant pressure of the entry of the liquid inside the leaf, in the point of the piston entering in the syringes, a coat of resilient material has been posed so to ensure the piston sliding in the syringe, but at the same time exerts a friction during said entry so that the infiltration liquid flows in a continuous, constant and slow way.

Is hence object of the invention a 1 ml syringe modified as above described.

Finally, in the transient transformation method of the present description also the solutions used have been modified with respect to those described in literature.
STATE OF THE ART CULTURE MEDIUM FOR INDUCTIVE COLTURE:
   LB bacterium culture medium supplemented with 150 μM of acetsyringon+MES 10 mM.
CULTURE MEDIUM FOR INDUCTIVE COLTURE OF THE DESCRIPTION:
   LB bacterium culture medium supplemented with 5 mM of acetsyringon+MES 100 μM.
INFILTRATION BUFFER DESCRIBED IN THE STATE OF THE

### ART:

MES buffer 10 mM+
acetosyringon 150 μM+
MgCl₂ 10 mM
   INFILTRATION BUFFER OF THE DESCRIPTION
MES buffer 10 mM+
Acetosyringon 25 μM+
MgCl₂ 25 mM

The lower concentration of acetosyringon used in this description allows Agrobacterium Tumefaciens to activate virulence genes but mildly. This allows, with respect to the state of the art, to avoid the necrosis of the leaf tissue to be infiltrated.

The higher optic density at 600 nm used in the present description, ensures that in the of infiltration solution volume used, there is a high number of bacterial cells containing the vector of interest.

In an embodiment the transient transformation method of the description may be described as follows:
a. inoculating a growth medium with at least a colony of Agrobacterium tumefaciens transformed with an expression vector capable of directing the expression of a recombinant protein of interest in plant and collecting through centrifugation and supernatant discarding cells from said Agrobacterium tumefaciens at a final OD₆₀₀ of growth of about 1.8-2.0;
b. re-suspending said cells in a buffer solution containing, for X ml of LB medium supplemented with suitable antibiotics, X/100 ml of MES buffer 10 mM at a pH of about 5.6, and X/2000 ml of acetosiryngone 10 mM, incubating said cells in said solution until obtaining a growth OD₆₀₀ of about 1.8-2.0 and collecting said cells in a pellet by centrifugation and discarding of the supernatant;
c. re-suspending the pellet thus obtained into an infiltration buffer containing MES 10 Mm at a pH of about 5,6 in a volume wherein the final OD600 is of about 1.8-2.0, MgCl2 in a final concentration of 25 mM and acetosiryngone at a final concentration of 25 μM, inserting the thus obtained final infiltration solution in a syringe without needle and exerting a pressure on the leaf lamina with said syringe transforming in a transient way the part of the leaf into which said solution diffuses.

The change above described allows obtaining a higher yield of recombinant protein production.

The transient transformation method of the present description, allows obtaining a recombinant protein expression from cells transformed by this method up to from 2 to 5 times higher with respect to the classic transient transformation method above described wherein:
the Agrobacterium transformed with the expression vector of interest culture OD₆₀₀ at the infiltration time is equal to 1, the culture medium for inductive is LB for bacteria culture supplemented with 150 μM of acetosyringon +MgCl₂ 10 mM.

Object of the invention is also, a kit for the transient transformation of plant tissues comprising one or more aliquots of:
a. a buffer solution containing, for X ml of LB medium supplemented with suitable antibiotics and X/100 ml of MES buffer 10 mM at a pH of about 5.6, X/2000 ml of acetosirygone 10 mM or one or more aliquots of each of said reagents at suitable concentrations;
b. One or more aliquots of a buffer solution containing for Xml of LB medium supplemented with suitable antibiotics, X/100 ml of buffer MES 10 mM ph of about 5.6, and X/2000 ml of acetosyringon 10 mM, or one or more parts of each of said reagents to at suitable concentrations. Said kit may further comprise one or more ml syringes without the needle support, at the end where the syringe content is expelled, a smooth hole of about 3 mm, and on the piston, at the site of entry in the syringe of said piston, a resilient coating. The kit buffers may also be provided in said kit, in a pre-mixed or non pre-mixed powder form, ready to re-suspend in suitable volumes of liquid as indicated in the kit instructions. The buffers may also be provided in part in powder form and part in liquid form. Any laboratory technician will be able to prepare the suitable powders or liquids suitable for the preparation of the buffers of the description. When ready for suspension, the powders of the kit of the description may be provided in flasks or bottles wherein the liquid volume to be reached may be marked thereon. Also the suspension liquid may be optionally provided in the kit, in pre-measured volumes suitable for the buffer preparation. The reagents may be provided in ready to use sterile containers.

The present description comprises also purification methods of the recombinant human protein pre-pro-NGF and of recombinant human protein NGF from transiently and stably transformed plants as described.

All methods comprise a first common step for the extraction of the total proteins wherein a particular tissue or the whole transformed plant as above described is pulverized in liquid nitrogen, incubate in extraction buffer, sonicated, and the soluble proteins are recovered by centrifugation.

The resulting solution is first purified in anionic exchange chromatography using a DEAE matrix followed by a second ionic (cationic) exchange chromatography using a SP-Sepharose matrix.

After the ionic exchange chromatography the pre-pro-NGF is purified by metal affinity chromatography.

After this step, it is possible to remove the C-terminal sequence.

To remove the elution buffer from the metal affinity chromatography solution (contained the purified pre-pro-NGF) the resulting solution is charged on a centrifugal filter, (such as amicon centricon) having a cut-off 5000 Kda to remove the imidazole present inside the solution with the purified pre-pro-NGF.

After this step the resulting solution is incubated overnight with TEV protease and than purified by metal affinity chromatography.

In the embodimente wherein the protein is produced with a tag facilitating the extraction, the extraction methods for the total proteins will be fast, unexpensive, and of easy industral scaling up due both to the natural biochemical characteristic of the protein (e.g. isoelectric point PI) and to the suitable ingeneering of the protein (by the addition of a C terminal tag such as His tag and of a protease recognition site such as TEV protease).

According to an embodiment, the purification of the pre-pro-NGF protein can be carried out according to two different methods.
a. extracting the total proteins from the transformed plant (sably or transiently) tissues expressing the exogenous protein of interest herein described, by grinding said tissues in liquid nitrogen;
b. the total protein extract thus obtained undergoes filtration with a centrifugal filter (e.g. amicon centricon) having a cut-off of 10 KDa, so to lower the ionic strength of the solution, to eliminate specific proteins having a molecular weight lower than 10 KDa and to concentrate (by a volume reduction) the remaining total proteins extracted as described above;
c. the filtrated thus obtained is purified by the presence of aspecific plant proteins by an anion exchange chromatography (inverse phase chromatography as the aspecific proteins are bound and the desired protein flows through) so to let the proteins having less affinity with the column, to directly elute in the flow-through; the pre-pro NGF, having an isoelectric point (IP) of about ph 9.94 has no affinity with the anion exchange matrix and pre-pro-NGF will flow-through during this step. Suitable matrix for this step are known to the skilled person and are by way of example but not limited thereto,
d. the flow through obtained in step c, comprising the pre-pro NGF expressed by the transformed plant of the description, is loaded as such on a cation exchange chromatography column (in this case direct phase chromatography as the protein of interest will tightly bind to the matrix) and the desired protein will be freed from the matrix by washing with a buffer solution having a pH between about 4 to about 7, to which a NaCl gradienti is added (from 0 to 1M); as cation exchangers the following, among other commonly known to the skilled person, may be used:: S-resin, sulfate derivatives; and CM resins, carboxylate derived mons, Sp-sepharose;
e. the extract thus obtained is further purified from aspecific plant proteins by affinity chromatography on a matrix functionalised with Nikel so that the recombinant protein, when fused with a His tag, can strongly bind to the resin and will be the last one eluted giving a purified product, and optionally, when vectors comprising nucleotide information for the transcription of Hist tag and TEV sequences bound to the pre-pro NGF protein as described above are used;
f. the TEV sequences are excised by the use of the TEV protease, the removal of the C terminal sequences is hence carried out by a further chromatographic step wherein an affinity chromatography on a matrix functionalised with nikel is carried out so to obtain a pre-pro NGF without the terminal TEV-KDEL-6×His tag. Alternatively, the step can be avoided and the method can be carried out with steps a, b, c, d, f.

The same method described above, comprising a further step g. can be carried out after step e. or f., to obtain the mature NGF form wherein the pre-pro sequences are removed by using a specific protease as described above (e.g. Matrix metalloproteinases, furine protease, plasmin protease).

The following examples are intended to teach some of the embodiments of the present descriprion without limiting the same.

### EXAMPLES

### Example 1

### Expression Vectors Production

The plant expression vectors for transient and stable expression of the protein pre-pro-NGF and NGF derive from the 2X35SCHT8466KP vector (Rainer Fisher) that has been modified by the insertion of a proteolytic site tobacco etch virus(TEV) and by the presence of a histidine tail for the affinity chromatography purification.

The insertion of these sequences has been obtained by the use of an oligo-nucleotide (sequence 8) containing the sequence for:
TEV+KDEL+6×his-Tag.

### Example 2

### Transient Transformation Agrobacterium Tumefaciens mediated with the NGF-vec-OLIGO vector

Transient transformation consists in the infiltration of plants leaf tissues mediated by the micro-organism Agrobacterium Tumefaciens transformed with the expression vector: NGF-vec-Oligo.

This kind of transformation allows to obtain a recombinant protein production localized only in the infiltration point and time-limited.

To carry out this kind of transformation a 1 ml syringe without needle is used, a pressure is applied on the leaf lamella so that the solution containing the Agrobacterium transformed with the expression vectors, diffuses into the leaf that will express the protein only in the infiltrated tissues for a limited time period.

The infiltration medium and the culture media preparation are widely described in literature (Tadeusz wrobleski et.al -Plant Biotechnology Journal vol.3, pp 259-273-2005; Jyoti Kapila et al -Plant science vol.122, pp 101-108-1997).

In the first project stage the protocol reported I literature was precisely followed, in a second moment, with the aim of improving the production yields of recombinant pre-pro-NGf protein and NGF protein, the standard protocol was modified achieving a better production of heterologous protein. In particular the growth OD at 600 nm (OD₆₀₀ₙₘ) of the Agrobacterium tumefaciens culture, the growth medium composition, and the infiltration buffer composition, and the manual infiltration technique by a 1 ml syringe were modified.

The optimization of the above described parameters had, as consequence, an increase of the production yield of the recombinant protein.

The new growth conditions and the new infiltration technique have been tested using a vector containing a reporter gene, Green florescent protein (GFP), prior to be applied to the transient expression of pre-pro-NGF and NGF.

Moreover, in order to increase the recombinant protein accumulation time of in transiently transformed tissues, the transformation is performed using also a suppressor of the post transcriptional silencing. Hence, the invention suggested shows an improvement of the agro-infiltration technique by the addition of some important changes to the standard protocols. These changes allow the obtaining of a higher yield of recombinant protein production. In the procedure herein developed of importance is the growth and the infiltration OD at 600 nm of the Agrobacterium, the development plant stage and the infiltration procedure by the use of 1 ml syringe without needle.

Materials:

Agrobacterium Tumefaciens strains used: GV3101 pmp90 RK and GV3101 pmp.

Culture medium: LB (Luria Bertani)
Antibiotics: rifamicine 50 mg/ml, gentamicine 25 mg/ml, Kanamicine 50 mg/ml for the strain GV3101 pmp90 RK.
Rifamicine 50 mg/ml, gentamicine 25 mg/ml for the strain GV3101 pmp 90.
Carbenicillin 50 mg/ml for the vector NGF-vec-OLIGO
Kanamicine 50 mg/ml for the vector containing the p19 post transcriptional gene silencing suppressor.

Plasmids used
NGF-vec-OLIGO
P19 post transcriptional gene silencing suppressor (Baulcombe)

Infiltration buffer:
MES buffer 10 mM ph=5.6
MgCl₂ 0.5 M
Acetosyringon 10 mM

Plant
Nicotiana benthamiana

Method

Preparation to the transient transformation with strain GV3101 pmp 90 RK with the vector NGF-vec-OLIGO and of strain pmp 90 with vector p19:

In order to obtain a higher yield of protein it is necessary to start from a pre-refreshed plate containing GV3101 pmp 90 RK+NGF-vec OLIGO and GV3101 pmp 90+p19.

Pre-inoculation

An inoculation of a colony of GV3101 pmp 90 RK+NGF-vec-oligo and GV3101 pmp 90+p19 derived from the pre-refreshed plate is carried out in LB medium supplemented with suitable antibiotics.

The inoculated colonies are grown at 28° C., 180 rpm up to a final growth OD at 600nm of about 1.8-2.0.

Inductive inoculation

500μl of pre-inoculation are inoculated in a solution containing 25 ml of LB medium with suitable antibiotics+250 μl of MES buffer ph 5.6+12.5 μl of acetosyringon 10 mM.

Incubation is carried out at 28° C., 180 rpm up to an OD 600 nm of 1.8-2.0.

Infiltration buffer

The cells GV3101 pmp 90 RK+NGF-vec-OLIGO and GV3101 pmp 90+p19 are precipitated at 4500 rpm, 4° C., and 30 minutes.

Supernatant is discarded and cells are re-suspended in a buffer containing: MES 10 mM ph 5.6 in a volume suitable to obtain a final OD at 600 nm of 2.0.

The following is added:
MgCl₂ 0.5 M (final concentration 25 mM)
Acetosyringon 10 mM (final concentration 25 μM)

The buffers containing Agrobacterium Tumefaciens with NGF-vec-Oligo and p19 are pooled in a ratio 1:1. The infiltration buffer is left for 3 hour at room temperature or overnight at 4° C. so that the acesyringon can activate the virulence genes necessary to the Agrobacterium for transferring to the plant cells the gene cassette containing the gene for the production of the pre-pro-NGF protein and NGF protein.

Preparation of Nicotiana Benthamiana plants for the transient transformation

Nicotiana benthamiana plants to be agro-infiltrated must be at the fourth leaf development stage cotyledon not included. The fast and uniform diffusion of the infiltration buffer inside the leaf tissue is made easier if the plants are wet 30 minutes prior to proceed to the transformation. Well hydrated plants have the leaf lamella lifted and this easies the entry of the Agrobacterium inside the plant cells.

Transformation

The transformation is carried out through a 1 ml syringe without the support for needle insertion (3 mm hole) and supplied with a resilient material on the piston in order to increase the friction of the piston inside the syringe.

For each transformed leaf about 1 ml of infiltration buffer containing GV3101 pmp90 RK+NGF-vec-Oligo and Gv3101 pmp 90+p19 is inoculated. Using a modified syringe and charged with 1 ml of buffer is performed a constant pressure in the lower portion of leaf lamina.

It is very important that the leaves are not excessively damaged, indeed, better results are obtained if the inoculation is limited to a maximum of two entrance points for the buffer in the leaves.

Sampling of the transient transformed plants

The sampling of the plants transient transformed with GV3101 pmp 90 Rk+NGF-vec- oligo and GV3101 pmp 9030 p19 is carried out 7 days post infiltration, time wherein highest accumulation of recombinant pre-pro-NGF protein is obtained.

Analysis of the plants transiently transformed with the vector NGF-vec-Oligo.

The genomic DNA of the leaves transiently transformed with the vector NGF-vec-OLIGO in the presence of p19 was analysed by PCR carried out using 3 primer pairs:
p35S forward (Seq ID 13) and pNGF reverse (Seq ID 10)

This primer pair is useful to detect the presence, inside the genome of the transformed plant cells, of the part of gene cassette containing the promoter 35S and the NGF gene.
pNGF forward (Seq ID 9) and pNGF reverse (Seq ID 10)

This primer pair is useful to detect the presence, inside the genome of the transformed plant cells, of the gene coding for the pre-pro-NGfF and NGF protein.
pNGf-bsph-forward (Seq ID 11) and pNGF-sal-reverse (Seq ID 12)

This primer pair is useful to detect the presence, inside the genome of the transformed plant cells, of the gene coding for the pre-pro-NGF protein and for the NGF protein and of the sequences placed at the C-terminal of the protein (TEV-KDEL-6×Hist-tag).

These PCR analyses are useful to confirm the insertion of the gene cassette containing the gene for the production of pre-pro-NGF and of NGF inside the genome of infiltrated cells.

On the same leaves an RT-PCR was carried out to verify the presence of transcript.

From the same leaves the total proteins were extracted which were analysed for the presence of pre-pro-NGf and NGF by the qualitative/quantitative test ELISA (NGF Eₘₐₓ Immunoassay system—promega)

Comparison between the standard procedure and the procedure modified according to the present description. Prior to carry on to the application of the new method studies on a reporter gene were, green florescent protein (GFP) was carried out (FIG. 1).

In a second moment, the standard procedure was compared to the modified procedure using a buffer with Agrobacterium Tumefaciens engineered with the vector NGF-vec-OLIGO. The effectiveness of the two protocols was compared in term of expression level of the recombinant protein measured by the analytic quantitative technique of the ELISA test (FIG. 2).

### Example 3

### Stable transformation of Nicotiana Benthamiana mediated by Agrobacterium Tumefaciens.

MATERIALS:

Medium for “in vitro” growth
TMS: MS (murrashighe Skoog) supplemented with 0.1 mg/ml of NAA (naftalenacetic hormone)+1 mg/L BA (benziladenin hormone)+sucrose ph 7.5+gelrite
YEP: peptone 10 g/l+yeast extract 10 g/l +NaCl 5 g/l ph 7
TRM: MS (murrashighe Skoog) supplemented with 0.1 mg/ml of NAA (naftalenacetic hormone)+Sucrose+gelrite.
Co-culture medium: MS (murrashighe Skoog) supplemented with 0.1 mg/ml of NAA (naftalenacetic hormone)+1 mg/L BA (benziladenin hormone)+sucrose, ph 7.5.

METHODS

Some portions having a side dimension of 0.5-1 cm are cut of from mature leaves of Nicotiana benthamiana and the explants are posed for 1 day in a plate containing TSM medium.

The Agrobacterium Tumefaciens containing the plant expression vector is grown overnight at 28° C. in YEP medium supplemented with the suitable antibiotics.

The Agrobacterium cells are centrifuged at 4500 rpm for 30 minutes and suspended in the co-culture medium up to an OD 600 nm of 0.6-1.

The explants are incubated for 5 minutes with the co-culture medium containing Agrobacterium.

The explants are washed with deionised water.

The explants are cultivated in TSM medium.

After 5 days the explants are transferred to a new TSM medium containing suitable antibiotics.

Once the callus is formed it is transferred in TRM medium up to the formation of a root.

After the root is grown the plants are transferred in a green house.

From this point on the selection of the plant (positive plants) containing the expression cassette of interest begins.

The positive plants are auto-pollinated upon obtaining a line stable for the presence of the expression cassette.

### Example 4

### Extraction of pre-pro-NGF from plants transformed transiently or stably with the vector NGF-vec-OLIGO.

The purification of the recombinant pre-pro-NGF-and NGF human protein derived from Nicotiana benthamiana stably transformed (GAPIone) and transiently transformed, is carried out through the use of fast, economic, and of easy industrial scaling up techniques using both the natural biochemical characteristics of the protein and the suitable engineering of the same (presence of C-terminal His-tag, presence of a recognition site for tobacco etch virus-TEV protease).

Total proteins extraction protocol Materials:

Extraction buffer for total proteins from plants stably and transiently transformed with the gene pre-pro-NGF
Extraction buffer=Phosphate buffer (NaH₂PO₄) 50 mM+NaCl 0.3M+DTT(dichlorodiphenyltrichloroethane) 0.1 mM+0.1%

Tween 20+100 μL of protease inhibitor cocktail (Thermo scientific) for each 100 ml of extraction buffer.

Buffers for ionic exchange chromatography.
Equilibration buffer:
   Pi 40 mM+NaCl 40 mM pH 7.5.
DEAE(diethylamminoethil) column cm 4×6, vol tot 75 ml
Sp-sepharose column
   cm 5.5×1.5, vol tot 9.7 ml.
Washing buffer:
   Pi 40 mM+NaCl 40 mM pH 7.5.
Elution buffer:
   Pi 75 mM pH 7.5, NaCl 450 mM
Metal affinity chromatography column: 2.5×1 cm
Metal affinity chromatography equilibration buffer: Pi 50 pH 7.85+NaCl 300 mM
His-tag elution buffer: Pi 75 mM pH 7.5, NaCl 450 mM+50mM imidazole, ph 6.
Protease TEV buffer: Pi pH 7.9 40 mM, 0.3 mM, DTT 1 mM,TEV 30 units, total volume 0.25 ml, temp 20° C.
Affinity chromatography After TEV: 0.50 ml di Ni-NTA equilibrated in Pi 40 mM, NaCl 45 mM pH 7.85

Extraction of total proteins from plant stably and transiently transformed with the gene pre-pro-NGF:

Nicotiana benthamiana plants transiently or stably transformed are grinded in liquid nitrogen upon obtaining a thin powder.

1 mL of the extraction buffer per gram of grinded plant is added and incubation 4° C. for 30 minutes is carried out.

2 sonication cycles of 10 minutes, in ice with ultrasounds (sonication amplitude 80%) are carried out.

A centrifugation at 30000 rpm for 30 minutes with subsequent recovery the supernatant in which the total soluble proteins are contained follows.

A second centrifugation round is carried out at 45000 rpm for 20 minutes and the supernatant is recovered.

The solution containing the total soluble protein is than concentrated and filtered using a centrifugal filter such as an Amicon centricon with a cut -off about 10 Kda.

The resulting solution is firstly loaded in an anion exchange chromatography column (eDEAE) equilibrated with the suitable equilibration buffer (Pi 40 mM+NaCl 40 mM pH 7.5.). The flow-trough obtained, containing the pre-pro-NGF is then loaded on a cation exchange chromatography (sp-Sepharose). After a prolonged washing step using the washing buffer (Pi 40 mM+NaCl 40 mM pH 7.5.) the pre-pro-NGF is eluted using the elution buffer (Pi 75 mM pH 7.5, NaCl 450 mM).

After the steps above, the resulting solution containing the pre-pro-NGF is loaded on a metal affinity chromatography column (nickel matrix).

The column is equilibrated using a Metal affinity chromatography equilibration buffer (Pi 50 pH 7.85+NaCl 300 mM).

The human recombinant pre-pro-NGF is finally eluted using an elution buffer such as His-tag elution buffer

(Pi 75 mM pH 7.5, NaCl 450 mM+50 mM imidazole, ph 6).

When present, the C-terminal sequence added as explained above can be removed using the protease TEV.

Before to proceed with this step is necessary to remove the imidazole present in the purified pre-pro-NGF buffer after the metal affinity chromatography, this step can be carried out using an amicon centricon 5000 Kda.

The pre-pro-NGF resulting from the centrifugal filter step is incubated overnight with the TEV buffer (Pi pH 7.9 40 mM, 0.3 mM, DTT 1 mM, TEV 30 units, total volume 0.25 ml, temp 20° C.).The solution is subsequently loaded on an Affinity chromatography After TEV column (0.50 ml di Ni-NTA) and equilibrated in Pi 40 mM, NaCl 45 mM pH 7.85.

The flow-through thus obtained contains the cut pre- pro-NGF is hence collected while the C-terminal sequence is removed from the affinity column using a Phosphate buffer (50 mM, NaCl 300 mM, Imidazolo 50 mM pH 6).

Analysis of plants transiently or stably transformed with the vector NGF-vec-OLIGO.

The genomic DNA of the plants transformed transiently or stably with the vector NGF-vec-OLIGO was analysed by PCR carried out by using 3 primer pairs:
p35S forward (Seq ID 13) and pNGF reverse (Seq ID 10)

This prime pair is useful to detect the presence inside the genome of the transiently or stably transformed plant of the presence of the part of the gene cassette containing the promoter 35S and the NGF gene.
pNGF forward (Seq ID 9) and pNGF reverse (Seq ID 10)

This primer pair is useful to detect the presence, inside the genome of the plant transiently or stably transformed, of the gene coding for the pre-pro-NGfF and NGF protein.
pNGf-bsph-forward (Seq ID 11) and pNGF-sal-reverse (Seq ID 12)

This primer pair is useful to detect the presence inside the genome of the plant transiently or stably transformed, of the gene coding for the pre-pro-NGfF and NGF protein and of the sequences placed at the C-terminal of the protein (TEV-KDEL-6×Hist-tag).

These PCR analyses are useful to confirm the effective insertion of the gene cassette containing the gene for the production of pre-pro-NGF and NGF inside the genome of transformed cells.

On the same plants a RT-PCR was carried out to verify the transcript presence.

From the same plants total proteins were extracted, which were analysed for the presence of pre-pro-NGf and NGF using the qualitative/quantitative test ELISA (NGF Eₘₐₓ Immunoassay system-promega).

DESCRIPTION OF THE SEQUENCES

SEQ ID NO: 1

| | |
|---|---|
| atg tcc atg ttg ttc tac act ctg atc aca gct ttt ctg atc ggc ata | 48 |
| cag gcg gaa cca cac tca gag agc aat gtc cct gca gga cac acc atc | 96 |
| ccc caa gtc cac tgg act aaa ctt cag cat tcc ctt gac act gcc ctt | 144 |
| cgc aga gcc cgc agc gcc ccg gca gcg gcg ata gct gca cgc gtg gcg | 192 |
| ggg cag acc cgc aac att act gtg gac ccc agg ctg ttt aaa aag cgg | 240 |
| cga ctc cgt tca ccc cgt gtg ctg ttt agc acc cag cct ccc cgt gaa | 288 |
| gct gca gac act cag gat ctg gac ttc gag gtc ggt ggt gct gcc ccc | 336 |
| ttc aac agg act cac agg agc aag cgg tca tca tcc cat ccc atc ttc | 384 |
| cac agg ggc gaa ttc tcg gtg tgt gac agt gtc agc gtg tgg gtt ggg | 432 |
| gat aag acc acc gcc aca gac atc aag ggc aag gag gtg atg gtg ttg | 480 |
| gga gag gtg aac att aac aac agt gta ttc aaa cag tac ttt ttt gag | 528 |
| acc aag tgc cgg gac cca aat ccc gtt gac agc ggg tgc cgg ggc att | 576 |
| gac tca aag cac tgg aac tca tat tgt acc acg act cac acc ttt gtc | 624 |
| aag gcg ctg acc atg gat ggc aag cag gct gcc tgg cgg ttt atc cgg | 672 |
| ata gat acg gcc tgt gtg tgt gtg ctc agc agg aag gct gtg aga aga | 720 |
| gcc tga | 726 |
| SEQ ID NO: 2 | |
| Met Ser Met Leu Phe Tyr Thr Leu Ile Thr Ala Phe Leu Ile Gly Ile | |
| Gln Ala Glu Pro His Ser Glu Ser Asn Val Pro Ala Gly His Thr Ile | |
| Pro Gln Val His Trp Thr Lys Leu Gln His Ser Leu Asp Thr Ala Leu | |
| Arg Arg Ala Arg Ser Ala Pro Ala Ala Ala Ile Ala Ala Arg Val Ala | |
| Gly Gln Thr Arg Asn Ile Thr Val Asp Pro Arg Leu Phe Lys Lys Arg | |
| Arg Leu Arg Ser Pro Arg Val Leu Phe Ser Thr Gln Pro Pro Arg Glu | |
| Ala Ala Asp Thr Gln Asp Leu Asp Phe Glu Val Gly Gly Ala Ala Pro | |
| Phe Asn Arg Thr His Arg Ser Lys Arg Ser Ser Ser His Pro Ile Phe | |
| His Arg Gly Glu Phe Ser Val Cys Asp Ser Val Ser Val Trp Val Gly | |
| Asp Lys Thr Thr Ala Thr Asp Ile Lys Gly Lys Glu Val Met Val Leu | |
| Gly Glu Val Asn Ile Asn Asn Ser Val Phe Lys Gln Tyr Phe Phe Glu | |
| Thr Lys Cys Arg Asp Pro Asn Pro Val Asp Ser Gly Cys Arg Gly Ile | |
| Asp Ser Lys His Trp Asn Ser Tyr Cys Thr Thr Thr His Thr Phe Val | |
| Lys Ala Leu Thr Met Asp Gly Lys Gln Ala Ala Trp Arg Phe Ile Arg | |
| Ile Asp Thr Ala Cys Val Cys Val Leu Ser Arg Lys Ala Val Arg Arg | |
| Ala | |
| SEQ ID NO: 3 | |
| Met Ser Met Leu Phe Tyr Thr Leu Ile Thr Ala Phe Leu Ile Gly Ile | |
| Gln Ala | |
| SEQ ID NO: 4 | |
| Glu Pro His Ser Glu Ser Asn Val Pro Ala Gly His Thr Ile Pro Gln | |
| Ala His Trp Thr Lys Leu Gln His Ser Leu Asp Thr Ala Leu Arg Arg | |
| Ala Arg Ser Ala Pro Ala Ala Ala Ile Ala Ala Arg Val Ala Gly Gln | |
| Thr Arg Asn Ile Thr Val Asp Pro Arg Leu Phe Lys Lys Arg Arg Leu | |
| Arg Ser Pro Arg Val Leu Phe Ser Thr Gln Pro Pro Arg Glu Ala Ala | |
| Asp Thr Gln Asp Leu Asp Phe Glu Val Gly Gly Ala Ala Pro Phe Asn | |
| Arg Thr His Arg Ser Lys Arg | |
| SEQ ID NO: 5 | |
| aagcttgcat gcctgcaggt ctcagaagac cagagggcta ttgagacttt tcaacaaagg | 60 |
| gtaatatcgg gaaacctcct cggattccat tgcccagcta tctgtcactt catcgaaagg | 120 |
| acagtagaaa aggaagatgg cttctacaaa tgccatcatt gcgataaagg aaaggctatc | 180 |
| gttcaagaat gcctctaccg acagtggtcc caaagatgga cccccaccca cgaggaacat | 240 |
| cgtggaaaaa gaagacgttc caaccacgtc ttcaaagcaa gtggattgat gtgataacat | 300 |
| ggtggagcac gacactctcg tctactccaa gaatatcaaa gatacagtct cagaagacca | 360 |
| gagggctatt gagacttttc aacaaagggt aatatcggga aacctcctcg gattccattg | 420 |
| cccagctatc tgtcacttca tcgaaaggac agtagaaaag gaagatggct tctacaaatg | 480 |
| ccatcattgc gataaaggaa aggctatcgt tcaagaatgc ctctaccgac agtggtccca | 540 |
| aagatggacc cccacccacg aggaacatcg tggaaaaaga agacgttcca accacgtctt | 600 |
| caaagcaagt ggattgatgt gatatctcca ctgacgtaag ggatgacgca caatcccact | 660 |
| atccttcgca agacccttcc tctatataag gaagttcatt tcatttggag aggacctcga | 720 |
| gaattcacaa cacaaatcag atttatagag agatttataa aaaaaaaaaa acatatggag | 780 |
| tggagctgga tctttctctt tctcctctca ggaactgcag gtgttcactc catgaccatg | 840 |
| ttgttctaca ctctgatcac agcttttctg atcggcatac aggcggaacc acactcagag | 900 |
| agcaatgtcc ctgcaggaca caccatcccc caagtccact ggactaaact tcagcattcc | 960 |
| cttgacactg cccttcgcag agcccgcagc gccccggcag cggcgatagc tgcacgcgtg | 1020 |
| gcggggcaga cccgcaacat tactgtggac cccaggctgt ttaaaaagcg gcgactccgt | 1080 |
| tcaccccgtg tgctgtttag cacccagcct ccccgtgaag ctgcagacac tcaggatctg | 1140 |
| gacttcgagg tcggtggtgc tgcccccttc aacaggactc acaggagcaa gcggtcatca | 1200 |
| tcccatccca tcttccacag gggcgaattc tcggtgtgtg acagtgtcag cgtgtgggtt | 1260 |
| ggggataaga ccaccgccac agacatcaag ggcaaggagg tgatggtgtt gggagaggtg | 1320 |
| aacattaaca acagtgtatt caaacagtac ttttttgaga ccaagtgccg ggacccaaat | 1380 |
| cccgttgaca gcgggtgccg gggcattgac tcaaagcact ggaactcata ttgtaccacg | 1440 |
| actcacacct ttgtcaaggc gctgaccatg gatggcaagc aggctgcctg gcggtttatc | 1500 |
| cggatagata cggcctgtgt gtgtgtgctc agcaggaagg ctgtgagaag agccgtcgac | 1560 |
| gaaaatcttt attttcaagg agtcgacaag gatgaacttc atcaccatca tcaccattaa | 1620 |
| ctcgaggggt agtcaagatg cataataaat aacggattgt gtccgtaatc acacgtggtg | 1680 |
| cgtacgataa cgcatagtgt ttttccctcc acttaaatcg aagggttgtg tcttggatcg | 1740 |
| cgcgggtcaa atgtatatgg ttcatataca tccgcaggca cgtaataaag cgaggggttc | 1800 |
| gaatcccccc gttacccccg gtaggggccc aggtaccggc gcgcctctag agtccgcaaa | 1860 |
| aatcaccagt ctctctctac aaatctatct ctctctattt ttctccagaa taatgtgtga | 1920 |
| gtagttccca gataagggaa ttagggttct tatagggttt cgctcatgtg ttgagcatat | 1980 |
| aagaaaccct tagtatgtat ttgtatttgt aaaatacttc tatcaataaa atttctaatt | 2040 |
| cctaaaacca aaatccagtg acctgcaggc atgcaagctc ctagctagca tcgac | 2095 |
| SEQ ID NO: 6 | |
| aagcttgcat gcctgcaggt ctcagaagac cagagggcta ttgagacttt tcaacaaagg | 60 |
| gtaatatcgg gaaacctcct cggattccat tgcccagcta tctgtcactt catcgaaagg | 120 |
| acagtagaaa aggaagatgg cttctacaaa tgccatcatt gcgataaagg aaaggctatc | 180 |
| gttcaagaat gcctctaccg acagtggtcc caaagatgga cccccaccca cgaggaacat | 240 |
| cgtggaaaaa gaagacgttc caaccacgtc ttcaaagcaa gtggattgat gtgataacat | 300 |
| ggtggagcac gacactctcg tctactccaa gaatatcaaa gatacagtct cagaagacca | 360 |
| gagggctatt gagacttttc aacaaagggt aatatcggga aacctcctcg gattccattg | 420 |
| cccagctatc tgtcacttca tcgaaaggac agtagaaaag gaagatggct tctacaaatg | 480 |
| ccatcattgc gataaaggaa aggctatcgt tcaagaatgc ctctaccgac agtggtccca | 540 |
| aagatggacc cccacccacg aggaacatcg tggaaaaaga agacgttcca accacgtctt | 600 |
| caaagcaagt ggattgatgt gatatctcca ctgacgtaag ggatgacgca caatcccact | 660 |
| atccttcgca agacccttcc tctatataag gaagttcatt tcatttggag aggacctcga | 720 |
| gaattcacaa cacaaatcag atttatagag agatttataa aaaaaaaaaa acatatggag | 780 |
| tggagctgga tctttctctt tctcctctca ggaactgcag gtgttcactc catgaccatg | 840 |
| ttgttctaca ctctgatcac agcttttctg atcggcatac aggcggaacc acactcagag | 900 |
| agcaatgtcc ctgcaggaca caccatcccc caagtccact ggactaaact tcagcattcc | 960 |
| cttgacactg cccttcgcag agcccgcagc gccccggcag cggcgatagc tgcacgcgtg | 1020 |
| gcggggcaga cccgcaacat tactgtggac cccaggctgt ttaaaaagcg gcgactccgt | 1080 |
| tcaccccgtg tgctgtttag cacccagcct ccccgtgaag ctgcagacac tcaggatctg | 1140 |
| gacttcgagg tcggtggtgc tgcccccttc aacaggactc acaggagcaa gcggtcatca | 1200 |
| tcccatccca tcttccacag gggcgaattc tcggtgtgtg acagtgtcag cgtgtgggtt | 1260 |
| ggggataaga ccaccgccac agacatcaag ggcaaggagg tgatggtgtt gggagaggtg | 1320 |
| aacattaaca acagtgtatt caaacagtac ttttttgaga ccaagtgccg ggacccaaat | 1380 |
| cccgttgaca gcgggtgccg gggcattgac tcaaagcact ggaactcata ttgtaccacg | 1440 |
| actcacacct ttgtcaaggc gctgaccatg gatggcaagc aggctgcctg gcggtttatc | 1500 |
| cggatagata cggcctgtgt gtgtgtgctc agcaggaagg ctgtgagaag agccgtcgac | 1560 |
| ggaggtggag gttctgcggc cgctcgtgga tctgagaaag atgagctcta aactcgaggg | 1620 |
| gtagtcaaga tgcataataa ataacggatt gtgtccgtaa tcacacgtgg tgcgtacgat | 1680 |
| aacgcatagt gtttttccct ccacttaaat cgaagggttg tgtcttggat cgcgcgggtc | 1740 |
| aaatgtatat ggttcatata catccgcagg cacgtaataa agcgaggggt tcgaatcccc | 1800 |
| ccgttacccc cggtaggggc ccaggtaccg gcgcgcctct agagtccgca aaaatcacca | 1860 |
| gtctctctct acaaatctat ctctctctat ttttctccag aataatgtgt gagtagttcc | 1920 |
| SEQ ID NO: 7 | |
| aagcttgcat gcctgcaggt ctcagaagac cagagggcta ttgagacttt tcaacaaagg | 60 |
| gtaatatcgg gaaacctcct cggattccat tgcccagcta tctgtcactt catcgaaagg | 120 |
| acagtagaaa aggaagatgg cttctacaaa tgccatcatt gcgataaagg aaaggctatc | 180 |
| gttcaagaat gcctctaccg acagtggtcc caaagatgga cccccaccca cgaggaacat | 240 |
| cgtggaaaaa gaagacgttc caaccacgtc ttcaaagcaa gtggattgat gtgataacat | 300 |
| ggtggagcac gacactctcg tctactccaa gaatatcaaa gatacagtct cagaagacca | 360 |
| gagggctatt gagacttttc aacaaagggt aatatcggga aacctcctcg gattccattg | 420 |
| cccagctatc tgtcacttca tcgaaaggac agtagaaaag gaagatggct tctacaaatg | 480 |
| ccatcattgc gataaaggaa aggctatcgt tcaagaatgc ctctaccgac agtggtccca | 540 |
| aagatggacc cccacccacg aggaacatcg tggaaaaaga agacgttcca accacgtctt | 600 |
| caaagcaagt ggattgatgt gatatctcca ctgacgtaag ggatgacgca caatcccact | 660 |
| atccttcgca agacccttcc tctatataag gaagttcatt tcatttggag aggacctcga | 720 |
| gaattcacaa cacaaatcag atttatagag agatttataa aaaaaaaaaa acatatggag | 780 |
| tggagctgga tctttctctt tctcctctca ggaactgcag gtgttcactc catgaccatg | 840 |
| ttgttctaca ctctgatcac agcttttctg atcggcatac aggcggaacc acactcagag | 900 |
| agcaatgtcc ctgcaggaca caccatcccc caagtccact ggactaaact tcagcattcc | 960 |
| cttgacactg cccttcgcag agcccgcagc gccccggcag cggcgatagc tgcacgcgtg | 1020 |
| gcggggcaga cccgcaacat tactgtggac cccaggctgt ttaaaaagcg gcgactccgt | 1080 |
| tcaccccgtg tgctgtttag cacccagcct ccccgtgaag ctgcagacac tcaggatctg | 1140 |
| gacttcgagg tcggtggtgc tgcccccttc aacaggactc acaggagcaa gcggtcatca | 1200 |
| tcccatccca tcttccacag gggcgaattc tcggtgtgtg acagtgtcag cgtgtgggtt | 1260 |
| ggggataaga ccaccgccac agacatcaag ggcaaggagg tgatggtgtt gggagaggtg | 1320 |
| aacattaaca acagtgtatt caaacagtac ttttttgaga ccaagtgccg ggacccaaat | 1380 |
| cccgttgaca gcgggtgccg gggcattgac tcaaagcact ggaactcata ttgtaccacg | 1440 |
| actcacacct ttgtcaaggc gctgaccatg gatggcaagc aggctgcctg gcggtttatc | 1500 |
| cggatagata cggcctgtgt gtgtgtgctc agcaggaagg ctgtgagaag agccgtcgac | 1560 |
| aaggatgaac ttcatcacca tcatcaccat taactcgagg ggtagtcaag atgcataata | 1620 |
| aataacggat tgtgtccgta atcacacgtg gtgcgtacga taacgcatag tgtttttccc | 1680 |
| tccacttaaa tcgaagggtt gtgtcttgga tcgcgcgggt caaatgtata tggttcatat | 1740 |
| acatccgcag gcacgtaata aagcgagggg ttcgaatccc cccgttaccc ccggtagggg | 1800 |
| cccaggtacc ggcgcgcctc tagagtccgc aaaaatcacc agtctctctc tacaaatcta | 1860 |
| tctctctcta tttttctcca gaataatgtg tgagtagttc ccagataagg gaattagggt | 1920 |
| tcttataggg tttcgctcat gtgttgagca tataagaaac ccttagtatg tatttgtatt | 1980 |
| tgtaaaatac ttctatcaat aaaatttcta attcctaaaa ccaaaatcca gtgacctgca | 2040 |
| ggcatgcaag ctcctagcta gcatcgac | 2068 |
| SEQ ID NO: 8 | |
| tcgacgaaaa tctttatttt caaggagtcg acaaggatga acttcatcac catcatcacc | 60 |
| attaactcga ggagct | 76 |
| SEQ ID NO: 9 | |
| cccaataaca gttttaccaa ggg | 23 |
| SEQ ID NO: 10 | |
| caggtcaggc tcttctcaca g | 21 |
| SEQ ID NO: 11 | |
| cctcatgacc atgttgttct acactctg | 28 |
| SEQ ID NO: 12 | |
| atagtcgacg gctcttctca cagccttcc | 29 |
| SEQ ID NO: 13 | |
| cactgacgta agggatgacg c | 21 |
| SEQ ID NO: 14 | |
| Glu Asn Leu Tyr Phe Gln Gly | |

1. A dicotyledon plant not grown for alimentary purposes or a plant tissue or a plant cell thereof which is stably transformed with an expression vector comprising a nucleic acid expression cassette under the control of a strong constitutive promoter, comprising a 5′UTR sequence, a nucleotide sequence coding for a plant leader sequence, a cDNA sequence coding for human pre-pro-NGF, a nucleotide sequence coding for a sequence that mediates the entry of a protein to which it is bound into the endoplasmic reticule, a 3′ UTR sequence, operatively linked, from 5′ to 3′, said plant being capable of constitutively expressing a human recombinant pre-pro-NGF.

2. The plant according to claim 1, wherein said cassette further comprises a nucleotide sequence coding for a purification tag positioned between said nucleotide sequence coding for a sequence that mediates the entry of a protein to which it is bound into the endoplasmic reticule and said 3′ UTR sequence.

3. The plant, according to claim 1, wherein said cassette further comprises a nucleotide sequence coding for a protease recognition site downstream the sequence coding for the pre-pro-NGF.

4. A portion Portions of the plant according to claim 1, which is selected from the group consisting of leaves, stems, flowers, seeds, roots, calluses, and protoplasts.

5. A method for stably transforming dicotyledon plants comprising the following steps:
cells from Agrobacterium tumefaciens are transformed with an expression vector comprising a nucleic acid expression cassette under the control of a strong constitutive promoter, comprising a 5′ UTR sequence, a nucleotide sequence coding for a plant leader sequence, a cDNA sequence coding for human pre-pro-NGF, a nucleotide sequence coding for a sequence that mediates the entry of a protein to which it is bound into the endoplasmic reticule, a 3′ UTR sequence, operatively linked, from 5′ to 3′;
leaf discs from said plant are transformed with a solution comprising said Agrobacterium tumefaciens cells;
formation of calluses from said leaf discs is induced;
the calluses thus obtained are selected for the presence of said vector;
the thus selected calluses are used for regenerating whole plants that are hemizygous for the cDNA coding for the human pre-pro-NGF; and
the thus obtained plants are allowed to self fertilise up to the third generation so to generate plants that are stably transformed with said vector that are capable of constitutively expressing human recombinant pre-pro-NGF.

6. The method according to claim 5, wherein said cassette said cassette further comprises a nucleotide sequence coding for a purification tag positioned between said nucleotide sequence coding for a sequence that mediates the entry of a protein to which it is bound into the endoplasmic reticule and said 3′ UTR sequence.

7. The method according to claim 5, wherein said cassette further comprises a nucleotide sequence coding for a protease recognition site downstream the sequence coding for the pre-pro-NGF.

8.-12. (canceled)

13. A method for the purification of rh pre-pro-NGF from dicotyledon plants or portions thereof transformed according to the method of claim 5 comprising the following steps:
a. extracting the total soluble proteins from said plant or portions through milling in liquid nitrogen, suspending them in a suitable buffer, sonicating, centrifuging and collecting the supernatant portion;
b. filtrating with centrifugal filters having a cut off of about 10 Kda collecting human pre-pro-NGF protein in the resulting filtrate;
c. the thus obtained filtrate is further purified by ionic (anion followed by cation) exchange chromatography;
d. the protein flow-through thus obtained is further purified by cation exchange chromatography and elution by NaCl gradient from 0 to 1 M; and, optionally,
e. the protein eluate obtained in d undergoes metal affinity chromatography on a resin matrix functionalised with the metal nickel.

14. The method according to claim 13, wherein said plants or portions thereof are transformed with a vector comprising a nucleotide sequence coding for a protease recognition site at the 3′ of a cDNA sequence coding for the recombinant human pre-pro-NGF further comprising the following step:
f. the protein flow through or eluate obtained in d. or e. is incubated with said protease and the digested protein is purified by affinity chromatography on a matrix with a resin functionalised with nickel.

15. A method for the purification of rh NGF from dicotyledon plants or portions thereof transformed according to the method of claim 5 comprising the following steps:
a′. extracting the total soluble proteins from said plant or portions through milling in liquid nitrogen, suspending them in a suitable buffer, sonicating, centrifuging and collecting the supernatant portion;
b′. filtrating with centrifugal filters having a cut off of about 10 Kda collecting human pre-pro-NGF protein in the resulting filtrate;
c′. the thus obtained filtrate is further purified by ionic (anion followed by cation) exchange chromatography;
d′. the protein flow-through thus obtained is further purified by cation exchange chromatography and elution by NaCI gradient from 0 to 1 M; and, alternatively,
e′. the protein eluate obtained in d undergoes metal affinity chromatography on a resin matrix functionalised with the metal nickel; or
f′. the protein flow through or eluate obtained in d. or e. is incubated with said protease and the digested protein is purified by affinity chromatography on a matrix with a resin functionalised with nickel, and
g′. removing said pre-pro sequence by using a protease such as a Matrix metalloproteinase, a furine protease, a plasmin protease.

16.-17. (canceled)

18. rh pre-pro-NGF obtainable by the method of claim 13.

19. rh pre-pro-NGF obtainable by the method of claim 14.

20. rh NGF obtainable by the method of claim 15.
